(19) United States
(12) Patent Application Publication
(10) Pub. No.: US 2026/0346014 A1
(43) Pub. Date: Nov. 5, 2026

(54) CEREBROVASCULAR ACCIDENT DETECTION USING SENSED NEURAL SIGNALS

(71) Applicant: Boston Scientific Neuromodulation Corporation, Valenicia, CA (US)

(72) Inventors: Luis Alfonso Castro Leiva, San Jose (CR); Daniel Antonio Vargas Campos, Alajuela (CR)

(21) Appl. No.: 19/655,503

(22) Filed: Apr. 22, 2026

Related U.S. Application Data
(60) Provisional application No. 63/799,997, filed on May 5, 2025.

(51) Int. Cl. A61N 1/36; A61B 5/00; A61B 5/0205; A61N 1/05

(52) U.S. Cl. CPC A61N 1/36103; A61B 5/0205; A61B 5/7267; A61B 5/7275; A61B 5/746; A61B 5/747; A61N 1/0529; A61N 1/36067

(57) ABSTRACT

A system may include an implantable medical device configured for implantation in a patient, a processing system and a communication system. The implantable medical device may include a set of electrodes from which to provide a stimulation electrode configuration and sensing electrode configuration, electrical stimulation circuitry configured to deliver a therapy by delivering electrical stimulation using the stimulation electrode configuration, and electrical sensing circuitry configured to use the sensing electrode configuration to sense electrical signals. The processing system may be configured to analyze the sensed electrical signals to determine a likelihood of ongoing or imminent cerebrovascular accident (CVA) and configured to provide a comparison of the determined likelihood to a predefined threshold. The processing system may be configured to use the communication system to communicate an alert based on the comparison of the determined likelihood to the predefined threshold.

### CLAIM OF PRIORITY

This application claims the benefit of U.S. Provisional Application No. 63/799,997, filed on May 5, 2025, which is hereby incorporated by reference in its entirety.

### TECHNICAL FIELD

This document relates generally to medical devices, and more particularly, to systems, devices and methods for identifying and warning of a potential cerebrovascular accident.

### BACKGROUND

Cerebrovascular accidents (CVAs), such as strokes or seizures, are medical emergencies where rapid response may improve patient outcomes. For example, timely medical intervention for a stroke can significantly enhance recovery chances and reduce the risk of long-term disabilities. Current detection methods are often limited to hospital settings, relying on technologies like MRI and CT scans. These technologies are often not available in emergency situations. There is a need to provide real-time detection and alerts for CVAs to enable faster medical responses.

Some papers have presented algorithms to predict and detect Ischemic Strokes in EEGs (see Choi, Y.-A.; Park, S.-J.; Jun, J.-A.; Pyo, C.-S.; Cho, K.-H.; Lee, H.-S.; Yu, J.-H. Deep Learning-Based Stroke Disease Prediction System Using Real-Time Bio Signals. Sensors 2021, 21, 4269. https://doi.org/10.3390/s21134269; and Badri, Cholid & Misbach, Jusuf & Soemardi, Tresna & Setiadji, Sutarmo. (2015). Electroencephalography (EEG) for detecting acute ischemic stroke. 42-48. 10.1109/ICICI-BME.2015.7401312). EEG tests are performed using external electrodes place on the scalp. Accuracy rates for predicting and detecting ischemic strokes in EEGs were greater than 90%.

### SUMMARY

Various embodiments of the present subject matter use sensed nerve activity that are sensed using implantable medical devices such as implantable SCS and implantable DBS devices to sense nerve activity and determine from the sensed nerve activity a likelihood of ongoing or imminent cerebrovascular accident (CVA). Alerts may be communicated to enable early intervention when it is determined that the likelihood is assessed to be significant (e.g., above a threshold).

It is expected that the effect of the CVA event (e.g., stroke or seizure) on brain activity can be detected from local field potentials (LFPs) within the brain sensed using the DBS leads. As the DBS leads are within the brain, it is expected that the LFP sensing capabilities of the DBS lead may be able to determine a likelihood of ongoing or imminent CVA even better than what was achieved for predicting and detecting strokes using external EEG electrodes sense EEGs. As the brain and the spinal cord are complexly integrated and form the core of the central nervous system, CVA events are also expected to cause detectible changes nerve activity in or near the spinal cord, which may be sensed as ecaps or other neural activity. Various embodiments are configured to adapt these implantable systems to be able to analyze the sensed electrical signals to determine a likelihood of ongoing or imminent cerebrovascular accident (CVA) and configured to determine when to communicate a CVA alert by comparing the determined likelihood to a predefined threshold.

A goal is to quickly identify when there is a possibility of ongoing or imminent CVA so that the patient can receive appropriate medical attention to diagnose and quickly respond to treat a medical condition. By way of example and not limitation, if the patient is diagnosed as likely having a stroke, the patient may be quickly administered a thrombolytic for breaking up blood clots and avoiding major brain damage. The system may be designed to monitor the sensed neural activity, and provide a warning to seek medical attention when it is determined that there is an unusual change in the sensed neural activity. The medical personnel can administer various tests to diagnose if a CVA event is occurring. According to some embodiments, the system may be designed to more accurately determine whether the unusual change is likely a CVA event. In some embodiments, the system may be able to discriminate whether the CVA event is a stroke or is a seizure and communicate that information to the medical personnel.

An example (e.g., “Example 1”) of a system may include an implantable medical device configured for implantation in a patient, a processing system and a communication system. The implantable medical device may include a set of electrodes from which to provide a stimulation electrode configuration and sensing electrode configuration, electrical stimulation circuitry configured to deliver a therapy by delivering electrical stimulation using the stimulation electrode configuration, and electrical sensing circuitry configured to use the sensing electrode configuration to sense electrical signals. The processing system may be configured to analyze the sensed electrical signals to determine a likelihood of ongoing or imminent CVA and configured to provide a comparison of the determined likelihood to a predefined threshold. The processing system may be configured to use the communication system to communicate an alert based on the comparison of the determined likelihood to the predefined threshold.

In Example 2, the subject matter of Example 1 may optionally be configured such that the CVA includes a stroke, and the therapy delivered using the electrical stimulation circuitry is not a therapy to either prevent or treat stroke.

In Example 3, the subject matter of Example 1 may optionally be configured such that the CVA includes a seizure, and the therapy delivered using the electrical stimulation circuitry is not a therapy to prevent or treat the seizure.

In Example 4, the subject matter of any one or more of Examples 1-3 may optionally be configured such that the set of electrodes is implanted in a brain and the implantable medical device is configured to provide a deep brain stimulation (DBS) therapy, and the electrical signals sensed by the electrical sensing circuitry include electrical signals in the brain.

In Example 5, the subject matter of Example 4 may optionally be configured such that the electrical signals sensed by the electrical sensing circuitry include local field potentials.

In Example 6, the subject matter of any one or more of Examples 4-5 may optionally be configured such that the DBS therapy includes a therapy for a movement disorder.

In Example 7, the subject matter of any one or more of Examples 4-5 may optionally be configured such that the DBS therapy includes a therapy for Tourette Syndrome, obsessive-compulsive disorder (OCD), depression, addiction, autism, anorexia nervosa, anxiety or schizophrenia.

In Example 8, the subject matter of any one or more of Examples1-3 may optionally be configured such that the implantable medical device is configured to deliver a spinal cord stimulation (SCS) therapy to treat chronic pain, the set of electrodes is implanted to stimulate a spinal cord or neural targets proximate to the spinal cord, and the electrical signals sensed by the electrical sensing circuitry include evoked compound action potentials from the spinal cord or the neural targets proximate to the spinal cord.

In Example 9, the subject matter of any one or more of Examples 1-8 may optionally be configured such that the system includes an external device, the processing system includes processing circuitry in the implantable medical device and in the external device, and the communication system includes wireless communication circuitry in the implantable medical device and wireless communication circuitry in the external device. The processing system and the communication system may be configured to enable the implantable medical device and the external device to communicate data corresponding to the likelihood or data used to determine the likelihood, and the processing system and the communication system may be configured to alert a user of the external device based on the comparison of the determined likelihood to the predefined threshold.

In Example 10, the subject matter of any one or more of Examples 1-8 may optionally be configured such that the system includes an external device and a cloud server. The processing system may include processing circuitry in the implantable medical device, in the external device, and in the cloud server. The communication system may include communication circuitry in the implantable medical device, in the external device, and in the cloud server. The processing system and the communication system may be configured to notify first responders, family members, or healthcare providers based on the comparison of the determined likelihood to the predefined threshold.

In Example 11, the subject matter of any one or more of Examples 1-10 may optionally be configured such that the implantable medical device further comprises an accelerometer configured to produce an accelerometer output signal. The processing system may be configured to analyze the accelerometer output signal with the sensed electrical signals to determine a likelihood of ongoing or imminent CVA.

In Example 12, the subject matter of any one or more of Examples 1-11 may optionally be configured such that the system further comprises at least one of a heart rate sensor, a blood pressure sensor or an oxygen sensor, and the processing system is configured to analyze an output of the at least one of the heart rate sensor, the blood pressure sensor or the oxygen sensor with the sensed electrical signals to determine a likelihood of ongoing or imminent CVA.

In Example 13, the subject matter of any one or more of Examples 1-12 may optionally be configured such that the processing system is configured to analyze the sensed signals by applying digital signal processing to the sensed electrical signals to determine the likelihood of ongoing or imminent CVA, or using a trained neural network to determine the likelihood of ongoing or imminent CVA.

In Example 14, the subject matter of any one or more of Examples 1-13 may optionally be configured such that the processing system is configured to analyze power changes in the sensed signals to determine the likelihood of ongoing or imminent CVA.

In Example 15, the subject matter of any one or more of Examples 12-14 may optionally be configured such that the processing system is configured to train a model to determine the likelihood of ongoing or imminent CVA. Training the model may include receiving signal data corresponding to sensed electrical signals from one or more patients, receiving ongoing or imminent CVA data for the one or more patients, determining patterns indicative of CVA including determining one or more features within the sensed electrical signals that are capable of being used to determine the likelihood of ongoing or imminent CVA, determining one or more feature-CVA relationships associating the one or more features within the sensed electrical signals to the likelihood of ongoing or imminent CVA, updating the one or more feature-CVA relationships based on additional signal data and additional CVA data, and adjusting the predefined threshold.

Example 16 includes subject matter (such as a method, means for performing acts, machine readable medium including instructions that when performed by a machine cause the machine to perform acts, or an apparatus to perform). The subject matter may be implemented using an implantable medical device having electrical stimulation circuitry, electrical sensing circuitry and a set of electrodes from which to provide a stimulation electrode configuration and a sensing electrode configuration. The subject matter may include delivering a therapy by delivering electrical stimulation using the electrical stimulation circuitry of the implantable medical device where the electrical stimulation is delivered using the stimulation electrode configuration, sensing electrical signals using the electrical sensing circuitry of the implantable medical device where the electrical signals are sensed using the sensing electrode configuration, using a processing system to analyze the sensed electrical signals to determine a likelihood of an ongoing or an imminent CVA and to compare the determined likelihood to a predefined threshold to provide a comparison, and communicating, using a communication system, an alert based on the comparison of the determined likelihood to the predefined threshold.

In Example 17, the subject matter of Example 16 may optionally be configured such that the CVA includes a stroke and the therapy is not a therapy to either prevent or treat stroke.

In Example 18, the subject matter of Example 16 may optionally be configured such that the CVA includes a seizure, and the therapy is not a therapy to either prevent or treat the seizure.

In Example 19, the subject matter of any one or more of Examples 16-18 may optionally be configured such that the therapy includes a DBS therapy and the electrical signals sensed using the electrical sensing circuitry includes local field potentials in a brain.

In Example 20, the subject matter of Example 19 may optionally be configured such that the DBS therapy includes a DBS therapy for a movement disorder.

In Example 21, the subject matter of any one or more of Examples16-18 may optionally be configured such that the therapy includes a SCS therapy and the electrical signals sensed using the electrical sensing circuitry includes evoked compound action potentials.

In Example 22, the subject matter of any one or more of Examples 16-21 may optionally be configured such that the system includes an external device, the processing system includes processing circuitry in the implantable medical device and in the external device, and the communication system includes wireless communication circuitry in the implantable medical device and wireless communication circuitry in the external device. The subject matter may include using the processing system and the communication system to communicate data corresponding to the likelihood or data used to determine the likelihood.

In Example 23, the subject matter of Example 22 may optionally be configured to use the processing system and the communication system to alert a user of the external device based on the comparison of the determined likelihood to the predefined threshold.

In Example 24, the subject matter of any one or more of Examples 22-23 may optionally be configured to use the processing system and the communication system to notify first responders, family members, or healthcare providers based on the comparison of the determined likelihood to the predefined threshold.

In Example 25, the subject matter of any one or more of Examples16-24 may optionally be configured to use the processing system to analyze an output of at least one of an accelerometer, a heart rate sensor, a blood pressure sensor or an oxygen sensor with the sensed electrical signals to determine a likelihood of ongoing or imminent CVA.

In Example 26, the subject matter of any one or more of Examples 16-25 may optionally be configured such that the sensed signals are analyzed by applying digital signal processing or using a trained neural network to the sensed electrical signals to determine the likelihood of ongoing or imminent CVA.

In Example 27, the subject matter of any one or more of Examples 16-26may optionally be configured such that the sensed signals are analyzed by analyzing power changes in the sensed signals to determine the likelihood of ongoing or imminent CVA.

In Example 28, the subject matter of any one or more of Examples 16-27may optionally be configured to include training a model to determine the likelihood of ongoing or imminent CVA. Training the model may include receiving signal data corresponding to sensed electrical signals from one or more patients, receiving ongoing or imminent CVA data for the one or more patients, determining patterns indicative of CVA including determining one or more features within the sensed electrical signals that are capable of being used to determine the likelihood of ongoing or imminent CVA, determining one or more feature-CVA relationships associating the one or more features within the sensed electrical signals to the likelihood of ongoing or imminent CVA, updating the one or more feature-CVA relationships based on additional signal data and additional CVA data, and adjusting the predefined threshold.

Example 29 includes subject matter (such as a method, means for performing acts, machine readable medium including instructions that when performed by a machine cause the machine to perform acts, or an apparatus to perform). The subject matter may be used for modifying a medical device system that includes a cloud server, an external device configured to communicate with the cloud server, and an implantable medical device configured to communicate with the external device. The implantable medical device may have electrical stimulation circuitry, electrical sensing circuitry and a set of electrodes from which to provide a stimulation electrode configuration and a sensing electrode configuration, wherein the implantable medical device is configured to deliver spinal cord stimulation (SCS) therapy to treat pain or is configured to deliver deep brain stimulation (DBS) to treat a movement disorder. The subject matter may include programming the medical device system to sense electrical signals using the electrical sensing circuitry and the sensing electrode configuration, programming the medical device system to analyze the sensed electrical signals to determine a likelihood of an ongoing or an imminent CVA, programming the medical device system to compare the determined likelihood to a predefined threshold to provide a comparison, and programming the medical device system to communicate, based on the comparison, an alert through at least one of the external device or the cloud server to notify first responders, family members, or healthcare providers.

In Example 30, the subject matter of Example 29 may optionally be configured to include updating firmware in the implantable medical device.

In Example 31, the subject matter of any one or more of Examples 29-30 may optionally be configured to include updating or installing an app in the external device.

In Example 32, the subject matter of any one or more of Examples 29-31 may optionally be configured such that the medical device system is programmed to perform digital signal processing to the sensed electrical signals to determine the likelihood of ongoing or imminent CVA.

In Example 33, the subject matter of any one or more of Examples 29-32 may optionally be configured such that the medical device system is programmed to use a trained neural network to determine the likelihood of ongoing or imminent CVA.

In Example 34, the subject matter of any one or more of Examples29-33 may optionally be configured such that the medical device system is programmed to analyze power changes in the sensed signals to determine the likelihood of ongoing or imminent CVA.

In Example 35, the subject matter of any one or more of Examples29-34 may optionally be configured such that the medical device system is programmed to analyze an output of at least one of an accelerometer, a heart rate sensor, a blood pressure sensor or an oxygen sensor, along with the sensed electrical signals, to determine a likelihood of ongoing or imminent CVA.

Example 36 includes subject matter that includes non-transitory machine-readable medium including instructions, which when executed by a machine, cause the machine to perform a method. The method may include, by way of example and not limitation, any of the subject matter for one or more of Examples 16-30. The machine-readable medium may include instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like. The term "machine-readable medium" is intended to include at least one machine-readable medium (e.g., two or more media which may be of the same type of media (such as but not limited to different nonvolatile semiconductor memory arrays) or different type of media (such as but not limited to a hard disk and a non-volatile semiconductor memory array). Furthermore, the term "machine" may include at least one processor, including one processor to implement all of the instructions, at least two processors where one processor operates on some of the instructions and other processor(s) operate on other instructions, or at least two processors where each processor is capable of operating on the same instructions. Thus, for example, distributed systems or systems with shared resources are contemplated.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various examples are illustrated by way of example in the figures of the accompanying drawings. Such examples are demonstrative and not intended to be exhaustive or exclusive examples of the present subject matter.

FIG. 1 illustrates, by way of example and not limitation, a medical device system for determining a likelihood of ongoing or imminent CVA and providing an alert.

FIG. 2 illustrates, by way of example and not limitation, a method for using a medical device system, such as the medical device system illustrated in FIG. 1, to determine a likelihood of ongoing or imminent CVA and to provide an alert.

FIG. 3 illustrates, by way of example and not limitation, a method for programming a medical device system with an implanted medical device to enable the medical device system to determine a likelihood of ongoing or imminent CVA and to provide an alert.

FIG. 4 illustrates, by way of example and not limitation, an electrical stimulation system, which may be used to deliver neurostimulation such as DBS or SCS.

FIG. 5 illustrates, by way of example and not limitation, an IPG for a DBS system.

FIGS. 6A-6B illustrate, by way of example and not limitation, leads that may be coupled for a DBS system.

FIGS. 7A-7F illustrate, by way of example and not limitation, various electrode arrangements that may be coupled to an IPG for an SCS system.

FIG. 8 illustrates, by way of example, an example of a medical device system for delivering an electrical therapy.

FIG. 9 illustrates, by way of example and not limitation, a system similar to the electrical therapy-delivery system of FIG. 8, which may be configured to provide DBS or SCS therapy(ies).

FIG. 10 illustrates, by way of example and not limitation, a neuromodulation device for delivering neuromodulation and for determining a likelihood of ongoing or imminent cerebrovascular accident (CVA) from sensed electrical signals and for communicating an alert.

FIG. 11 illustrates, by way of example and not limitation, an external system with sensing functions.

FIG. 12 is a flowchart depicting machine-learning pipeline, according to some examples.

FIG. 13 illustrates, by way of example and not limitation, a process for creating machine-learning models that may be used to determine a likelihood of an ongoing or an imminent CVA and to compare the determined likelihood to a predefined threshold for a CVA alert.

FIG. 14 illustrates, by way of example and not limitation, training of a machine-learning program to detect the CVA likelihood, according to some example embodiments.

FIG. 15 illustrates, by way of example and not limitation, use of a system configured to detect the CVA likelihood, according to some example embodiments.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized, and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

Medical devices may include therapy-delivery devices configured to deliver a therapy to a patient and/or monitors configured to monitor a patient condition via user input and/or sensor(s). For example, therapy-delivery devices for ambulatory patients may include wearable devices and implantable devices, and further may include, but are not limited to, stimulators (such as electrical, thermal, or mechanical stimulators). An example of a wearable device includes, but is not limited to, transcutaneous electrical neural stimulators (TENS), such as may be attached to glasses, an article of clothing, or a patch configured to be adhered to skin. Implantable stimulation devices may deliver electrical stimuli to treat various biological disorders, such spinal cord stimulators (SCS) to treat chronic pain, cortical and Deep Brain Stimulators (DBS) to treat motor and psychological disorders, Peripheral Nerve Stimulation (PNS), Functional Electrical Stimulation (FES), and other neural stimulators to treat urinary incontinence, sleep apnea, shoulder subluxation, etc. By way of example and not limitation, a DBS system may be configured to treat tremor, bradykinesia, and dyskinesia and other motor disorders associated with Parkinson’s Disease (PD). DBS therapy has been proposed to treat other conditions including dementia. DBS therapy has also been proposed for Tourette Syndrome, obsessive-compulsive disorder (OCD), depression, addiction, autism, anorexia nervosa, anxiety and schizophrenia. An example of a PNS system is a vagal nerve stimulation (VNS) system. VNS may include stimulation of the cervical vagus and/or may include stimulation of a branch of the vagus nerve such as the auricular nerve. VNS has been proposed as an external stimulator (e.g., TENs) over the auricular nerve, and has been proposed as an implanted device (e.g., cervical vagal nerve implant).

Medical devices, including the therapy delivery devices, may be engineered with the capability to sense neural signals. For example, implantable SCS devices will likely have the capacity to sense neural signals in the spine such compound action potentials and DBS devices will likely have the capacity to sense brain signals such as local field potentials. SCS and DBS devices are discussed below as specific examples, although it is expressly noted that PNS devices may also be designed with the capacity to sense neural signals in peripheral nerve(s), and other medical devices may monitor neural activity for other therapies or for diagnostic purposes.

Patients who have an implanted DBS or SCS device, or other medical device, may have a risk of CVA events such as a stroke or a seizure. Various embodiments of the present subject matter use the technology of the medical device to determine a likelihood of ongoing or imminent CVA and communicate an alert to the patient, caregivers, first responders (also referred to as emergency responders), and medical care providers. For example, the technology of the SCS device may be used to sense neural signals, such as evoked compound actions potentials (ecaps), in or near the spinal cord or the technology of the DBS device may be used to sense neural signals, such as local field potentials (LFPs), in the brain. The medical device system may be used to analyze the sensed neural signals to detect or otherwise determine a likelihood that a patient will experience an imminent CVA event (e.g., stroke or seizure) or is experiencing an ongoing CVA event. For example, it may be determined that the sensed neural signals from the spine or the brain are a precursor to a CVA. The sensed neural signals may be analyzed using different data analysis methods (for example, but not limited to artificial intelligence (AI), pattern recognition, digital signal processing (DSP), and the like).

The specific electrical signals may be affected by the location and severity of the CVA event, as well as the interconnectivity of the brain and the location of the implantable medical device. Various embodiments do not diagnose the CVA event. For example, the system may be designed to monitor the sensed neural activity and provide a warning to seek medical attention when it is determined that there is an unusual change in the sensed neural activity. As a specific example, signal power for specific frequencies may be determined and monitored, and a change in the power in the signal at certain frequencies that is greater than a threshold may indicate a potential CVA event. Other embodiments may be designed to more accurately discriminate among different CVA events (e.g., discriminate whether the CVA event is a stroke or is a seizure). The system may determine that there is a likelihood of a CVA event that is determined to be significant (e.g., over a threshold), and then provide an alarm when it is determined that there is a significant likelihood of the CVA event. The system need not have 100% certainty of the CVA event nor have an accurate diagnosis of the CVA event in order to provide the CVA event alarm. The caretakers, first responders or clinicians may quickly perform other evaluations and take other actions upon receiving the CVA event alert. For example, EEGs, CTs, or MRIs may be administered to further evaluate the patient with the potential CVA event. The cardiac activity (e.g., measured pulse and/or rate) may be used to evaluate the potential CVA event or may be used in addition to the sensed electrical signals to determine the likelihood of CVA events.

Implanted medical devices, such as implantable pulse generators (IPGs), may be configured to wirelessly connect to external device(s). By way of example, the external device(s) may include a phone, a tablet, or a watch, and the wireless communication technology used by the implantable device and external device(s) may include Bluetooth® such as Bluetooth Low Energy (BLE). These external device(s) may be configured to communicate over other wireless and/or wired communication networks. Examples of wireless communication networks may include cellular networks and WiFi networks. This connectivity allows the system to notify first responders, family members, or healthcare providers through secure cloud infrastructure, ensuring rapid response in emergency situations. The analysis of the signal may be performed using a distributed system, such that the analysis may be performed using any one or more of the implantable medical device, the external device(s) and/or the cloud servers. The system may be designed flexibly to adapt to varying levels of data complexity and processing needs and may adjust where the analysis is performed based on the required computational intensity.

The communication capabilities of these networks allow a CVA alert, indicating a likelihood of a current or imminent CVA event, to be sent to alert first responders, family or doctors. The CVA alert may be sent after a determined CVA likelihood reaches a value higher than a threshold. Thus, various embodiments address the need for real-time detection and alerts for CVAs by leveraging advanced sensing and data analysis technologies integrated into implantable medical devices such as implantable SCS devices and implantable DBS devices that are capable of sensing neural signals, such as local field potentials (LFPs) from the brain and evoked compound action potentials (ECAPs) from the spinal cord.

The present subject matter enhances the detection capabilities and provides a proactive solution to managing CVAs outside traditional medical facilities. For example, it is noted that older patients who may be more likely to have an implantable DBS device or an implantable SCS device may also have a greater risk of a stroke. Thus, even though the implanted DBS device or SCS device is not configured to treat or prevent a stroke or treat or prevent a seizure, that patient's DBS or SCS device may be configured (e.g., reprogrammed) to also provide CVA alerts (e.g., an alert of an ongoing or imminent stroke and/or ongoing or imminent seizure). The alert may correspond to a determination that a CVA event detection is likely to be occurring because the metric(s) of the CVA event are over a defined threshold. The neural signals (e.g., local field potentials or compound action potentials) may be analyzed using algorithms such as artificial intelligence, digital signal processing, and/or pattern recognition to assess the likelihood of an ongoing or imminent CVA. Notably, the system need not be configured to deliver a closed-loop CVA therapy, and the system need not diagnose CVA. Rather, the system may be configured to provide an early warning to facilitate timely medical intervention based on a likelihood or probability of a CVA event. As such, the system may be incorporated into an SCS device configured to treat pain or into a DBS device configured to treat a movement disorder. Additional sensors, such as accelerometers, to enhance detection capabilities. The distributed nature of the system's computational processes means that analysis can occur on the IPG, mobile device, or cloud servers, depending on the computational intensity required. This flexibility ensures that the system can adapt to varying levels of data complexity and processing needs.

Algorithm(s) may be implemented using DSP techniques to extract features of the sensed electrical signal to identify biomarkers or directly use raw data with neural networks. Raw data may be classified using neural networks such as a Convolutional Neural Network (CNN), a Long-Short Term Memory (LSTM) network, a transformer, or ensembles of neural networks. These neural networks are examples of artificial intelligence architectures that may be implemented to classify and determine the likelihood of CVA events. The algorithm(s) may be distributed such that the execution may be at the implantable medical device, the mobile device or the cloud servers.

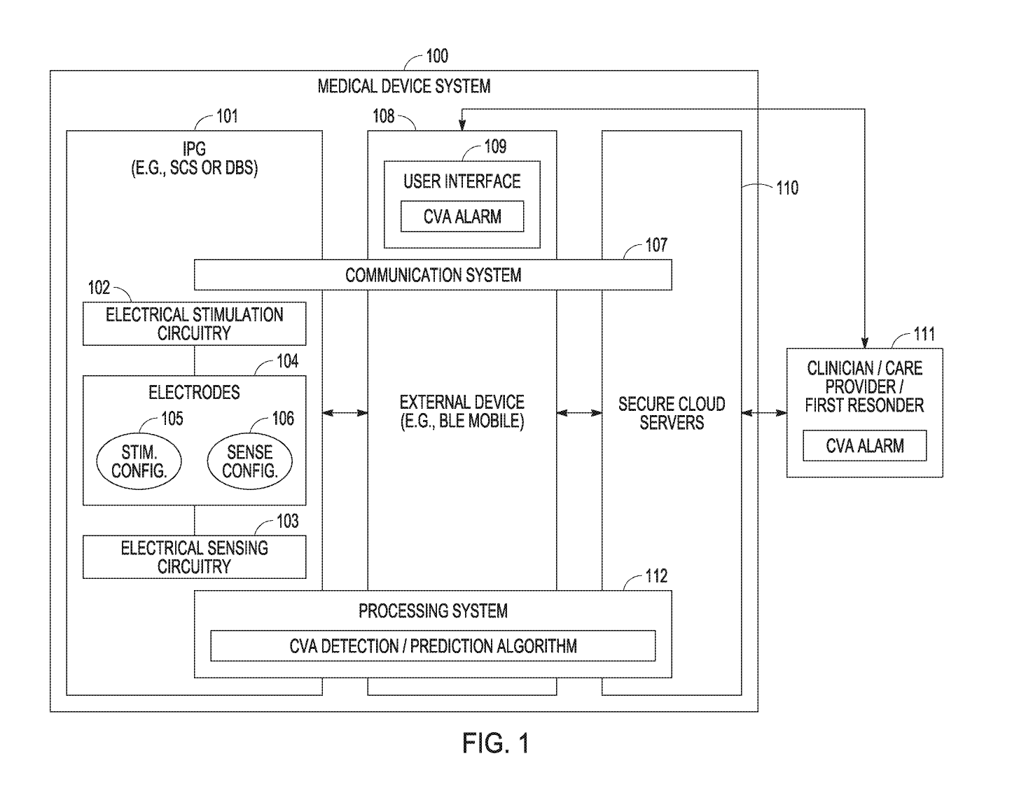

FIG. 1 illustrates, by way of example and not limitation, a medical device system for determining a likelihood of ongoing or imminent CVA and providing an alert. As illustrated, the system 100 may include an implantable pulse generator (IPG) 101. By way of example and not limitation, the IPG may be a spinal cord stimulator (SCS) or a deep brain stimulator (DBS). However, the medical device system 100 may include other pulse generators such as a transcutaneous electrical neural stimulators (TENS), Peripheral Nerve Stimulation (PNS), and Functional Electrical Stimulation (FES). The medical device system 100 may be configured or programmed to treat a condition. Thus, by way of example and not limitation, a DBS system may be configured to treat motor disorders such as, but not limited to, tremor, bradykinesia, and dyskinesia associated with Parkinson’s Disease (PD), and an SCS system may be configured to treat pain.

The IPG 101 may include electrical stimulation circuitry 102, electrical sensing circuitry 103, and a set of electrodes 104. The IPG may be configured to use select electrode(s) from the set of electrodes 104 as stimulation electrode(s) in a stimulation configuration 105 and may be configured to use select electrode(s) from the set of electrodes 104 as sensing electrode(s) in a sensing configuration 106. Electrodes within the set of electrodes 104 may be inactive or active for use in the stimulation configuration 105, and electrodes within the set of electrodes 104 may be inactive or active for use in the sensing configuration 106. Any of the electrode(s) may be both used in the stimulation configuration 105 and in the sensing configuration 106.

The electrical sensing circuitry and the sensing configuration of electrodes may be used to sense neural signals. For example, a DBS system may sense LFPs, or an SCS system may be configured to sense ecaps from the spinal cord. The electrical sensing circuitry may sense evoked resonant neural activity (ERNA) or other signals related to the electrical activity within the patient.

The medical device system 100 may include a communication system 107. The communication system may provide communication between the IPG and at least one external device 108, between the at least one external device 108 and secure cloud server(s), between secure cloud server(s) and at least one user device used by one or more of clinician(s), care provider(s), and/or a first responder(s). The communication system may be configured to enable the external device(s) to communicate directly to the user device(s) used by one or more of clinician(s), care provider(s), and/or a first responder(s).

The communication system 107 may be configured for use to transmit an alert signal to an external device 108, such as a BLE-enabled mobile device. For example, the alert signal may be transmitted when a likelihood of a CVA event exceeds a predefined threshold. The external device(s) 108 may include user interface(s) 109 that may output the CVA alarm. For example, the user interface(s) 109 may include a display, a speaker, and/or a haptic actuator, and may communicate the CVA alarm to a patient or other user using visual, audio and/or haptic signals. The external device 108 may communicate the alert to the secure cloud servers 110, which may then communicate the alert to a user device 111 used by the clinician, care provider or first responders which also may be referred to as emergency responders. The user device 111 may include a computer, phone or tablet used by a clinician, care provider or first responder. The external device 108 may communicate the alert to the user device 111 used by the clinician, care provider or first responders. The communication link(s) between the external device 108 and user device 111 may include network(s) such as cellular networks, local area networks, wide area networks, etc. For example, the external device 108 may be a smart phone capable of calling the user device 111.

A processing system 112 may be configured to analyze the sensed neural signals using one or more algorithms, illustrated as a CVA Detection / Prediction algorithm, to determine the likelihood of an ongoing or imminent CVA. The processing system 112 may include processing circuitry in the IPG 101. The processing system 112 may include processing circuitry in external device(s) 108, such as a mobile device like a phone or a tablet and/or may include processing circuitry in secure cloud server(s) 110. Function(s) of the processing system may be incorporated into any one of the IPG 101, external device 108 or secure cloud server(s) 110, or may be distributed across two or more of the IPG 101, external device(s) 108 or the secure cloud server(s) 110. The processing system may include cloud computing, fog computing, and/or edge computing. Cloud computing may include a network of devices or servers connected over the Internet. Cloud computing may have very large storage space and processing capabilities. However, cloud computing can have higher latencies. Fog computing occurs physically closer to the end user compared to centralized data centers. The infrastructure of fog computing may connect end devices with central servers in the cloud. Fog computing may provide lower latency for quicker responses and may use other communication technology other than the Internet. Edge computing is done at the device level. The processing for different functions may be distributed over multiple devices and may be distributed over edge computing, fog computing and cloud computing.

The IPG may be configured with other sensing capabilities, such as but not limited to accelerometers and sensors configured to detect cardiac activity (e.g., heart rhythm, heart rate, pulse, blood flow), muscle activity, respiration (e.g., respiration rate, respiration volume), activity / motion, and posture. The processing system may be configured to use at least some of these or other inputs to assist with analyzing the sensed electrical signal for the CVA detection and prediction.

In addition to communicating the alert, some embodiments may be configured to communicate additional data such as the raw signal data, extracted signal feature data, other sensor data, and the like, which may assist the medical providers with diagnosing the patient condition.

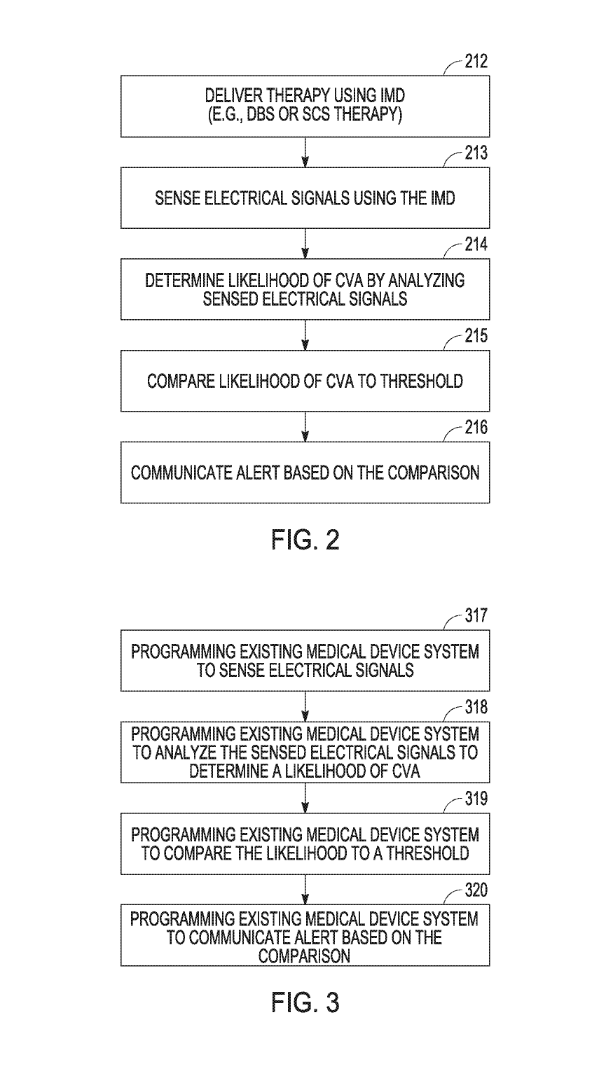

FIG. 2 illustrates, by way of example and not limitation, a method for using a medical device system, such as the medical device system illustrated in FIG. 1, to determine a likelihood of ongoing or imminent CVA and to provide an alert. As illustrated, the method may include delivering a therapy using the IMD 212. By way of example, the therapy may include a DBS or SCS therapy. As illustrated at 213, the method may include sensing electrical signals using sensing configuration of the device. At 214, the sensed neural signals may be analyzed using one or more algorithms to determine the likelihood of an ongoing or imminent CVA. A processing system, such as the processing system illustrated in FIG. 1, may perform this analysis. This likelihood of CVA may be compared to a predefined threshold at 215. If the likelihood exceeds the threshold, the communication system may transmit a CVA alert to a user of an external device or a CVA alert to a user device used by a clinician(s), care provider(s) or first responder(s). The transmission of the alert may occur automatically or semiautomatically when the likelihood exceeds the threshold. A fully automatic alert needs to further human intervention. A semiautomatic alert may be partially automatic and performed partially manually. An example of a partial manual performance may be an indicator (e.g., button on a display) queries the patient and then automatically performing a process based at least in part on the response.

FIG. 3 illustrates, by way of example and not limitation, a method for programming a medical device system with an implanted medical device to enable the medical device system to determine a likelihood of ongoing or imminent CVA and to provide an alert. The method may be performed on a previously-implanted device configured to deliver a therapy such as a DBS therapy or a SCS therapy. Thus, these DBS or SCS systems, which were not previously able to determine a likelihood of ongoing or imminent CVA and to provide an alert, are programmed to be able to provide CVA alerts. As illustrated, the method may include programming the existing system to sense neural signals 317. For example, an existing DBS system may be programmed to sense LFPs or an existing SCS system may be programmed to sense ECAPs. At 318, the existing medical device system may be programmed to analyze these sensed signals using one or more algorithms to determine the likelihood of a CVA. At 319, the existing medical device system may be programmed to compare this likelihood to a predefined threshold. The existing medical device system may be programmed to, upon exceeding the threshold, automatically or semiautomatically communicate an alert based on the comparison. The system may be designed to monitor the sensed neural activity, and provide a warning to seek medical attention when it is determined that there is an unusual change in the sensed neural activity. The medical personnel can administer various tests to diagnose if a CVA event is occurring. According to some embodiments, the system may be designed to more accurately determine whether the unusual change is likely a CVA event. In some embodiments, the system may be able to discriminate whether the CVA event is a stroke or is a seizure and communicate that information to the medical personnel.

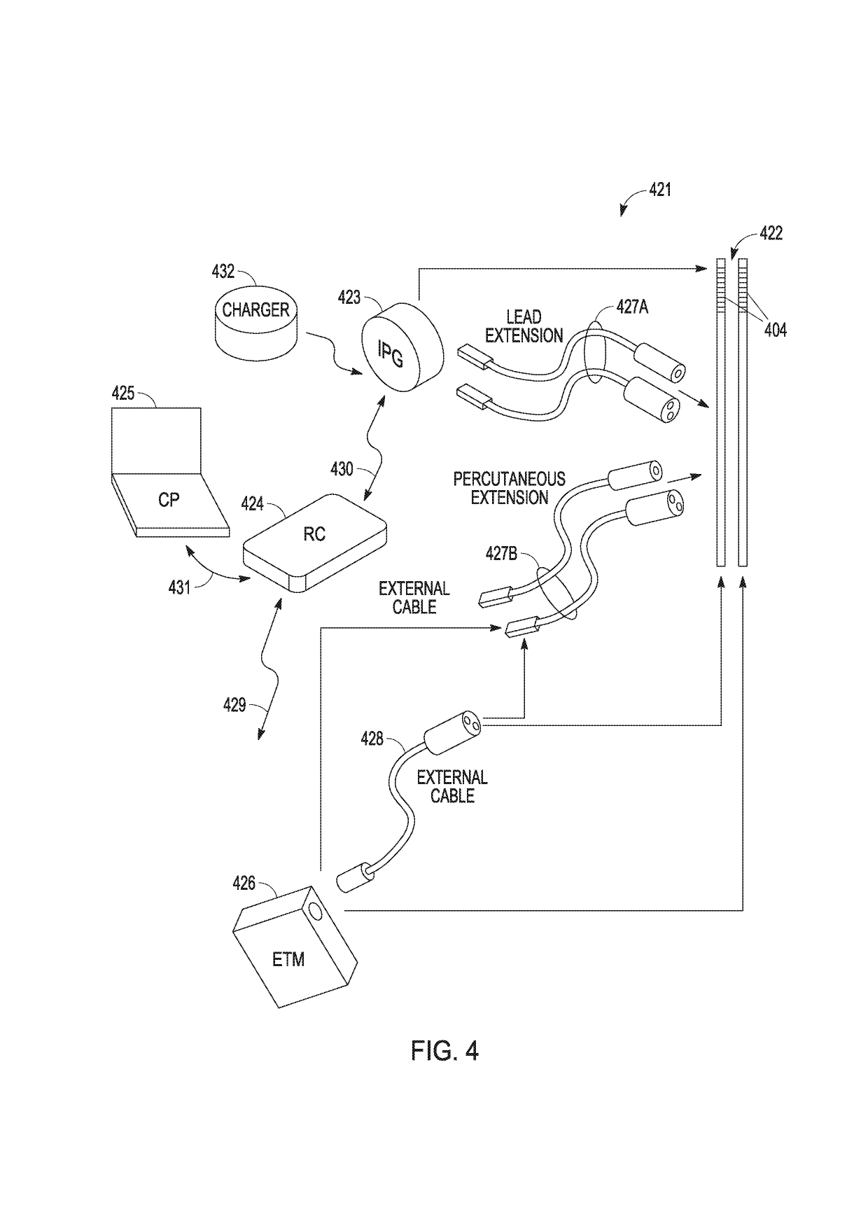

FIG. 4 illustrates, by way of example and not limitation, an electrical stimulation system 421, which may be used to deliver neurostimulation such as DBS or SCS. The electrical stimulation system 421 may generally include a one or more (illustrated as two) of implantable neuromodulation leads 422, a waveform generator such as an implantable pulse generator (IPG) 423, an external remote controller (RC) 424, a clinician programmer (CP) 425, and an external trial modulator (ETM) 426. The IPG 423 may be physically connected via one or more percutaneous lead extensions 427A to the neuromodulation lead(s) 422, which carry a plurality of electrodes 404. The electrodes, when implanted in a patient, form an electrode arrangement. As illustrated, the neuromodulation leads 422 may be percutaneous leads with the electrodes arranged in-line along the neuromodulation leads or about a circumference of the neuromodulation leads. Any suitable number of neuromodulation leads can be provided, including only one, as long as the number of electrodes is greater than two (including the IPG case function as a case electrode) to allow for lateral steering of the current. Other types of leads may be used. The IPG 423 includes pulse generation circuitry that delivers electrical modulation energy in the form of a pulsed electrical waveform (i.e.,a temporal series of electrical pulses) to the electrodes in accordance with a set of modulation parameters.

The ETM 426 may also be physically connected via the percutaneous lead extensions 427B and external cable 428 to the neuromodulation lead(s) 422. The ETM 426 may have similar pulse generation circuitry as the IPG 423 to deliver electrical modulation energy to the electrodes in accordance with a set of modulation parameters. The ETM 426 is a non-implantable device that may be used on a trial basis after the neuromodulation leads 422 have been implanted and prior to implantation of the IPG 423, to test the responsiveness of the modulation that is to be provided. Functions described herein with respect to the IPG 423 can likewise be performed with respect to the ETM 426.

The RC 424 may be used to telemetrically control the ETM 426 via a bi-directional RF communications link 429. The RC 424 may be used to telemetrically control the IPG 423 via a bi-directional RF communications link 430. Such control allows the IPG 423 to be turned on or off and to be programmed with different modulation parameter sets. The IPG 423 may also be operated to modify the programmed modulation parameters to actively control the characteristics of the electrical modulation energy output by the IPG 423. A clinician may use the CP 425 to program modulation parameters into the IPG 423 and ETM 426 in the operating room and in follow-up sessions.

The CP 425 may indirectly communicate with the IPG 423 or ETM 426, through the RC 424, via an IR communications link 431 or another link. The CP 425 may directly communicate with the IPG 423 or ETM 426 via an RF communications link or other link (not shown). The clinician detailed modulation parameters provided by the CP 425 may also be used to program the RC 424, so that the modulation parameters can be subsequently modified by operation of the RC 424 in a stand-alone mode (i.e., without the assistance of the CP 425). Various devices may function as the CP 425. Such devices may include portable devices such as a lap-top personal computer, mini-computer, personal digital assistant (PDA), tablets, phones, or a remote control (RC) with expanded functionality. Thus, the programming methodologies can be performed by executing software instructions contained within the CP 425. Alternatively, such programming methodologies can be performed using firmware or hardware. In any event, the CP 425 may actively control the characteristics of the electrical modulation generated by the IPG 423 to allow the desired parameters to be determined based on patient feedback or other feedback and for subsequently programming the IPG 423 with the desired modulation parameters. To allow the user to perform these functions, the CP 425 may include a user input device (e.g., a mouse and a keyboard), and a programming display screen housed in a case. In addition to, or in lieu of, the mouse, other directional programming devices may be used, such as a trackball, touchpad, joystick, touch screens or directional keys included as part of the keys associated with the keyboard. An external device (e.g., CP) may be programmed to provide display screen(s) that allow the clinician to, among other functions, select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), generate a pain map of the patient, define the configuration and orientation of the leads, initiate and control the electrical modulation energy output by the neuromodulation leads, and select and program the IPG with modulation parameters, including electrode selection, in both a surgical setting and a clinical setting. The external device(s) (e.g., CP and/or RC) may be configured to communicate with other device(s), including local device(s) and/or remote device(s). For example, wired and/or wireless communication may be used to communicate between or among the devices.

An external charger 432 may be a portable device used to transcutaneously charge the IPG 433 via a wireless link such as an inductive link 443. Once the IPG 423 has been programmed and its power source has been charged by the external charger or otherwise replenished, the IPG 423 may function as programmed without the RC 424 or CP 425 being present.

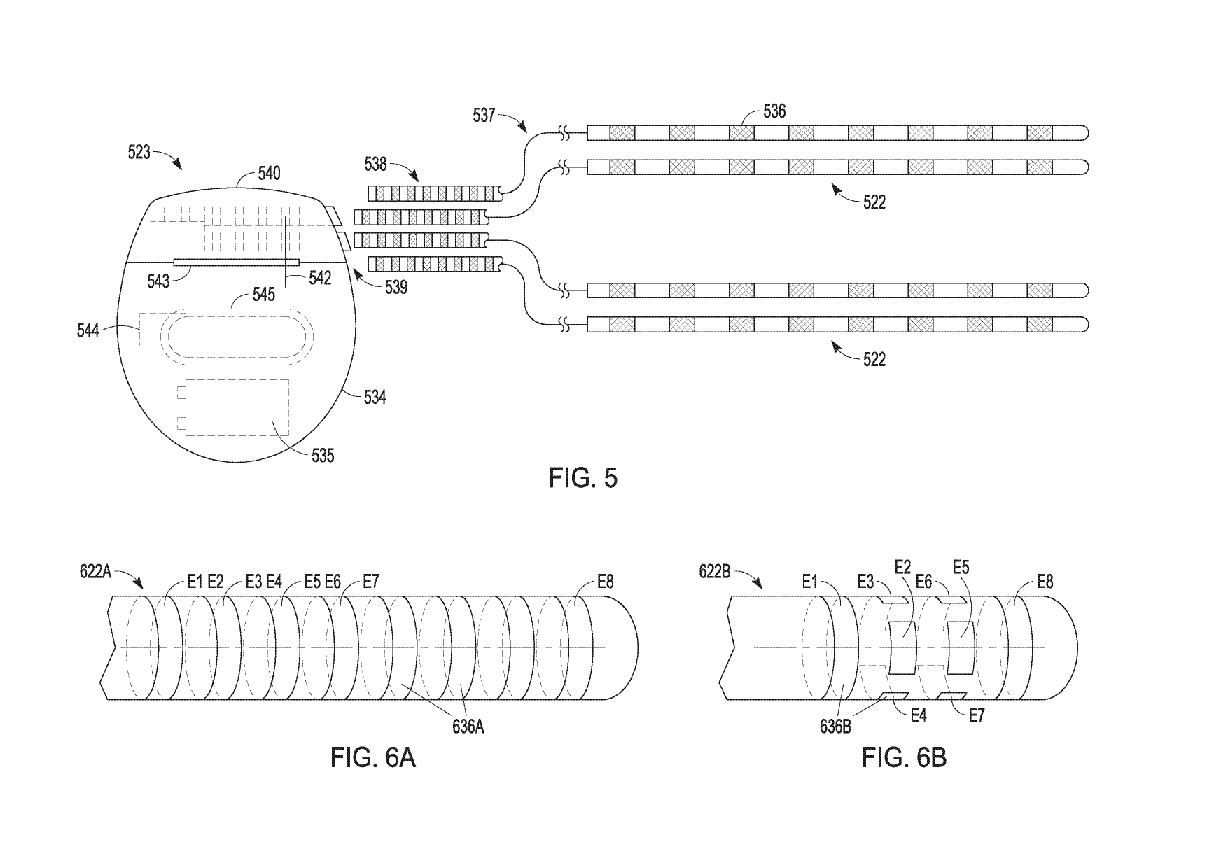

FIG. 5 illustrates, by way of example and not limitation, an IPG for a DBS system. The IPG 523, which is an example of the IPG 423 of the electrical stimulation system 421 as illustrated in FIG. 4, may include a biocompatible device case 534 that holds the circuitry and a battery 535 for providing power for the IPG 523 to function, although the IPG 523 can also lack a battery and can be wirelessly powered by an external source. The IPG 523 may be coupled to one or more leads, such as leads 522 as illustrated herein. The leads 522 can each include a plurality of electrodes 536 for delivering electrostimulation energy, recording electrical signals, or both. In some examples, the leads 522 can be rotatable so that the electrodes 536 can be aligned with the target neurons after the neurons have been located such as based on the recorded signals. The electrodes 536 can include one or more ring electrodes, and/or one or more sets of segmented electrodes (or any other combination of electrodes), examples of which are discussed below with reference to FIGS. 6A and 6B.

The leads 522 can be implanted near or within the desired portion of the body to be stimulated. In an example of operations for DBS, access to the desired position in the brain can be accomplished by drilling a hole in the patient’s skull or cranium with a cranial drill (commonly referred to as a burr), and coagulating and incising the dura mater, or brain covering. A lead can then be inserted into the cranium and brain tissue with the assistance of a stylet (not shown). The lead can be guided to the target location within the brain using, for example, a stereotactic frame and a microdrive motor system. In some examples, the microdrive motor system can be fully or partially automatic. The microdrive motor system may be configured to perform actions such as inserting, advancing, rotating, or retracing the lead.

Lead wires 537 within the leads may be coupled to the electrodes 536 and to proximal contacts 538 insertable into lead connectors 539 fixed in a header 540 on the IPG 523, which header can comprise an epoxy for example. Alternatively, the proximal contacts 538 may connect to lead extensions (not shown) which are in turn inserted into the lead connectors 539. Once inserted, the proximal contacts 538 connect to header contacts 541 within the lead connectors 539, which are in turn coupled by feedthrough pins 542 through a case feedthrough 543 to stimulation circuitry 544 within the case 534. The type and number of leads, and the number of electrodes, in an IPG is application specific and therefore can vary.

The IPG 523 can include an antenna 545 allowing it to communicate bi-directionally with a number of external devices. The antenna 545 may be a conductive coil within the case 534, although the coil of the antenna 545 may also appear in the header 540. When the antenna 545 is configured as a coil, communication with external devices may occur using near-field magnetic induction. The IPG 523 may also include a Radio-Frequency (RF) antenna. The RF antenna may comprise a patch, slot, or wire, and may operate as a monopole or dipole, and preferably communicates using far-field electromagnetic waves, and may operate in accordance with any number of known RF communication standards, such as Bluetooth, Zigbee, WiFi, Medical Implant Communication System (MICS), and the like.

In a DBS application, the IPG 523 is typically implanted under the patient’s clavicle (collarbone). The leads 522 (which may be extended by lead extensions, not shown) can be tunneled through and under the neck and the scalp, with the electrodes 216 implanted through holes drilled in the skull and positioned for desired target(s) for DBS therapy. The IPG 523 can also be implanted underneath the scalp closer to the location of the electrodes’ implantation. The leads 422, or the extensions, can be integrated with and permanently connected to the IPG 523 in other solutions.

Stimulation in IPG 523 is typically provided by pulses each of which may include one phase or multiple phases. For example, a monopolar stimulation current can be delivered between a lead-based electrode (e.g., one of the electrodes 536) and a case electrode. A bipolar stimulation current can be delivered between two lead-based electrodes (e.g., two of the electrodes 536). Stimulation parameters typically include current amplitude (or voltage amplitude), frequency, pulse width of the pulses or of its individual phases; electrodes selected to provide the stimulation; polarity of such selected electrodes, i.e., whether they act as anodes that source current to the tissue, or cathodes that sink current from the tissue. Each of the electrodes can either be used (an active electrode) or unused (OFF). When the electrode is used, the electrode can be used as an anode or cathode and carry anodic or cathodic current. In some instances, an electrode might be an anode for a period of time and a cathode for a period of time. These and possibly other stimulation parameters taken together comprise a stimulation program that the stimulation circuitry 544 in the IPG 523 can execute to provide therapeutic stimulation to a patient.

In some examples, a measurement device coupled to the muscles or other tissue stimulated by the target neurons, or a unit responsive to the patient or clinician, can be coupled to the IPG 523 or microdrive motor system. The measurement device, user, or clinician can indicate a response by the target muscles or other tissue to the stimulation or recording electrode(s) to further identify the target neurons and facilitate positioning of the stimulation electrode(s). For example, if the target neurons are directed to a muscle experiencing tremors, a measurement device can be used to observe the muscle and indicate changes in, for example, tremor frequency or amplitude in response to stimulation of neurons. Alternatively, the patient or clinician can observe the muscle and provide feedback.

FIGS. 6A-6B illustrate, by way of example and not limitation, leads that may be coupled to the for a DBS system. FIG. 6A shows a lead 622A with electrodes 636A disposed at least partially about a circumference of the lead 622A. The electrodes 636A may be located along a distal end portion of the lead. As illustrated herein, the electrodes 636A are ring electrodes that span 360 degrees about a circumference of the lead 622A. A ring electrode allows current to project equally in every direction from the position of the electrode, and typically does not enable stimulus current to be directed from only a particular angular position or a limited angular range around the lead. A lead which includes only ring electrodes may be referred to as a non-directional lead.

FIG. 6B shows a lead 622B with electrodes 636B including ring electrodes such as E1 at a proximal end and E8 at the distal end. Additionally, the lead 622B also include a plurality of segmented electrodes (also known as split-ring electrodes). For example, a set of segmented electrodes E2, E3, and E4 are around the circumference at a longitudinal position, each spanning less than 360 degrees around the lead axis. In an example, each of electrodes E2, E3, and E4 spans 90 degrees, with each being separated from the others by gaps of 30 degrees. Another set of segmented electrodes E5, E6, and E7 are located around the circumference at another longitudinal position different from the segmented electrodes E2, E3 and E4. Segmented electrodes such as E2-E7 can direct stimulus current to a selected angular range around the lead.

Segmented electrodes can typically provide superior current steering than ring electrodes because target structures in DBS or other stimulation are not typically symmetric about the axis of the distal electrode array. Instead, a target may be located on one side of a plane running through the axis of the lead. Through the use of a radially segmented electrode array, current steering can be performed not only along a length of the lead but also around a circumference of the lead. This provides precise three-dimensional targeting and delivery of the current stimulus to neural target tissue, while potentially avoiding stimulation of other tissue. In some examples, segmented electrodes can be together with ring electrodes. A lead which includes at least one or more segmented electrodes may be referred to as a directional lead. In an example, all electrodes on a directional lead can be segmented electrodes. In another example, there can be different numbers of segmented electrodes at different longitudinal positions.

Segmented electrodes may be grouped into sets of segmented electrodes, where each set is disposed around a circumference at a particular longitudinal location of the directional lead. The directional lead may have any number of segmented electrodes in a given set of segmented electrodes. By way of example and not limitation, a given set may include any number between two to sixteen segmented electrodes. In an example, all sets of segmented electrodes may contain the same number of segmented electrodes. In another example, one set of the segmented electrodes may include a different number of electrodes than at least one other set of segmented electrodes.

The segmented electrodes may vary in size and shape. In some examples, the segmented electrodes are all of the same size, shape, diameter, width or area or any combination thereof. In some examples, the segmented electrodes of each circumferential set (or even all segmented electrodes disposed on the lead) may be identical in size and shape. The sets of segmented electrodes may be positioned in irregular or regular intervals along a length the lead.

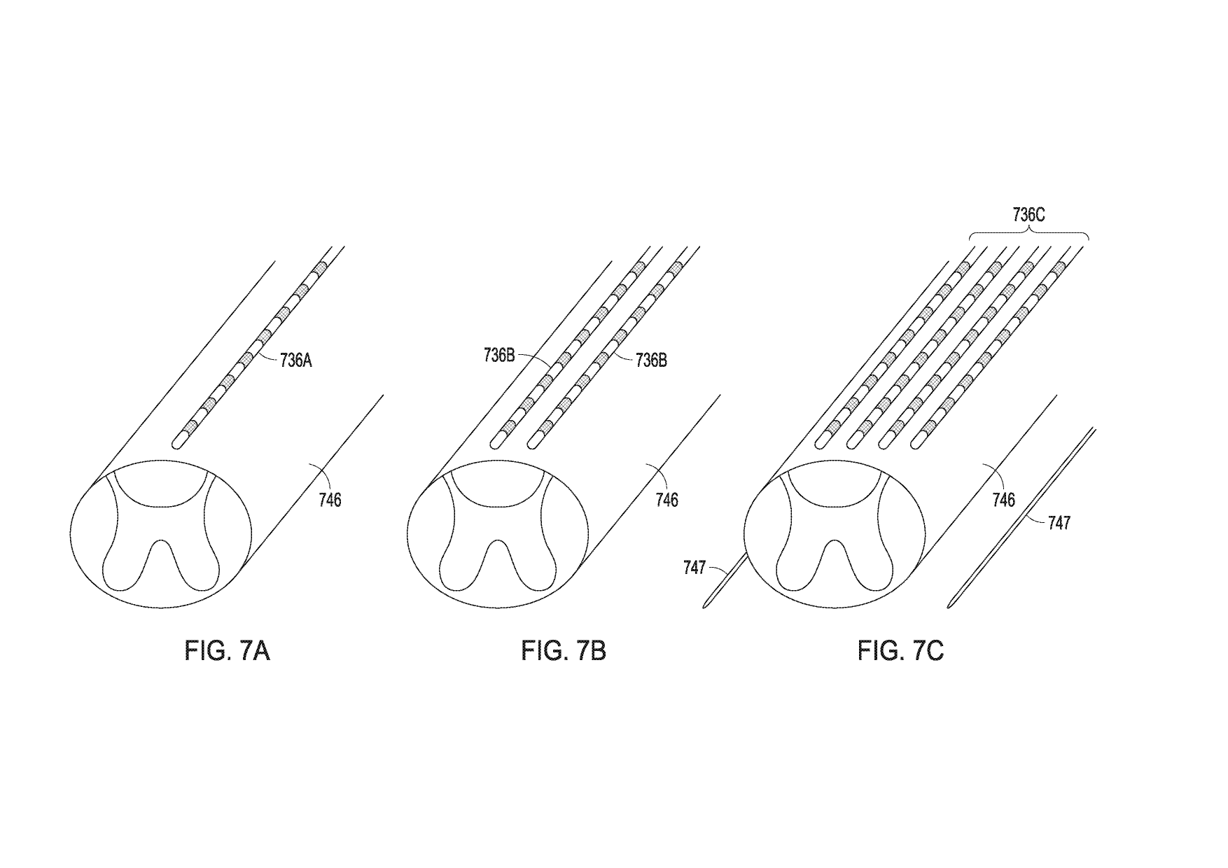

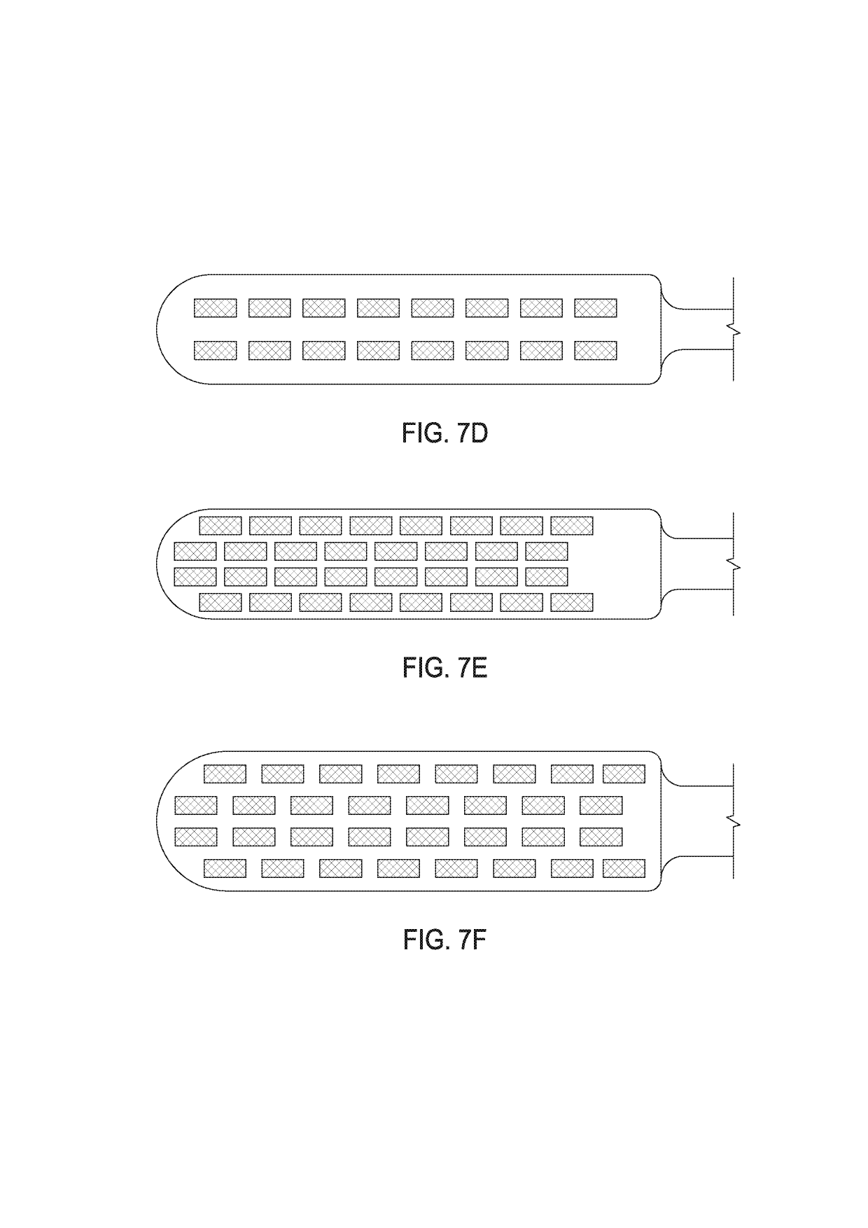

FIGS. 7A-7F illustrate, by way of example and not limitation, various electrode arrangements that may be coupled to an IPG for an SCS system. FIG. 7A is a schematic view of a single electrical modulation lead 736A implanted over approximately the longitudinal midline of the patient’s spinal cord 746. For example, FIG. 7B is a schematic view of two electrical modulation leads 736B implanted in the dorsal epidural space, and FIG. 7C is a schematic view of four electrical modulation leads 736C implanted in the dorsal epidural space. Some embodiments may position one or more lead(s) outside of the dorsal epidural space (e.g., in lateral or ventral epidural space and/or outside of the epidural space). The additional electrodes provide greater ability to focus a neuromodulation field to modulate one or more targeted nerve tracts or a targeted portion(s) of one or more nerve tracts. Any other plurality of leads or a multiple column paddle lead can also be used.

A ring electrode allows current to project equally in every direction from the position of the electrode, and typically does not enable stimulus current to be directed from only a particular angular position or a limited angular range around the lead. A lead which includes only ring electrodes may be referred to as a non-directional lead. However, the electrodes may include one or more ring electrodes, and/or one or more sets of segmented electrodes (or any other combination of electrodes). A lead which includes at least one or more segmented electrodes may be referred to as a directional lead. Some embodiments may non-directional or directional leads to stimulate the sympathetic chains 747.

Segmented electrodes may provide better current steering than ring electrodes. Through the use of a radially segmented electrode array, current steering may be performed not only along a length of the lead but also around a circumference of the lead. This may provide precise three-dimensional targeting and delivery of the current stimulus to neural target tissue, while potentially avoiding stimulation of other tissue. In some examples, segmented electrodes can be together with ring electrodes. In an example, there can be different numbers of segmented electrodes at different longitudinal positions. Segmented electrodes may be grouped into sets of segmented electrodes, where each set is disposed around a circumference at a particular longitudinal location of the directional lead. The directional lead may have any number of segmented electrodes in a given set of segmented electrodes. By way of example and not limitation, a given set may include any number between two to sixteen segmented electrodes. In an example, all sets of segmented electrodes may contain the same number of segmented electrodes. In another example, one set of the segmented electrodes may include a different number of electrodes than at least one other set of segmented electrodes. The segmented electrodes may vary in size and shape. In some examples, the segmented electrodes are all of the same size, shape, diameter, width or area or any combination thereof. In some examples, the segmented electrodes of each circumferential set (or even all segmented electrodes disposed on the lead) may be identical in size and shape. The sets of segmented electrodes may be positioned in irregular or regular intervals along a length the lead and/or around the circumference of the lead.

Paddle leads are another type of lead. FIGS. 7D-7F illustrate different arrangements of electrodes (illustrated as rectangles) on the paddles. The different electrode arrangements provided by these different lead examples, along with variations in the position of the of the electrodes with respect to the neural tissue, will have different sensing capabilities. The system may be designed to sense a baseline for the neural activity and detect when there is a significant change from that baseline.

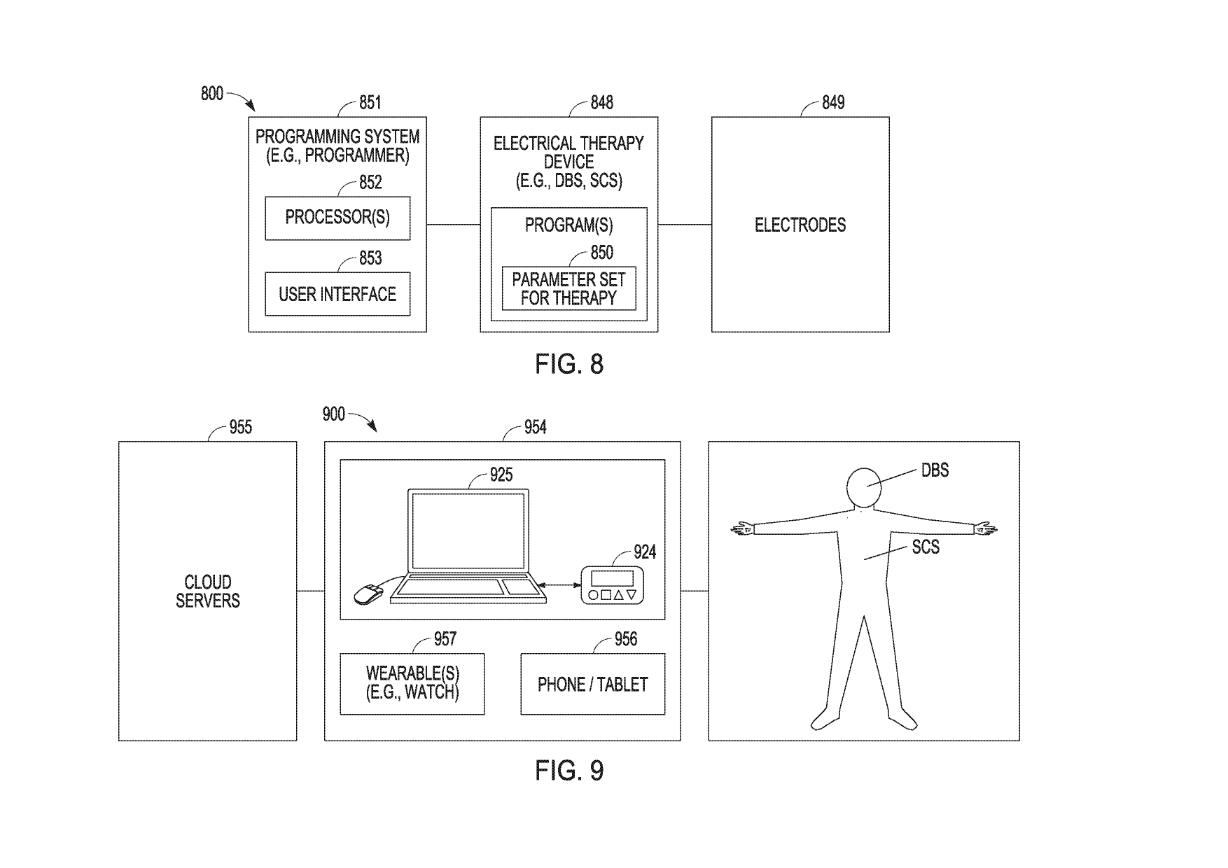

FIG. 8 illustrates, by way of example, an example of a medical device system for delivering an electrical therapy. The illustrated system 800 may include an electrical therapy device 848 configured to deliver an electrical stimulation to electrodes 849 to treat a condition in accordance with a programmed parameter set 850 for the therapy. The system 800 may include a programming system 851, which may function as at least a portion of a processing system, that may include one or more processors 852 and a user interface 853. The programming system 851 may be used to program and/or evaluate the parameter set(s) used to deliver the therapy. By way of example and not limitation, the illustrated system 800 may be a DBS system, a SCS system, or other neurostimulator system.

A therapy may be delivered according to a parameter set. The parameter set may be programmed into the device to deliver the specific therapy using specific values for a plurality of therapy parameters. For example, the therapy parameters that control the therapy may include pulse amplitude, pulse frequency, pulse width, and electrode configuration (e.g., selected electrodes, polarity and fractionalization). The parameter set includes specific values for the therapy parameters. The number of electrodes available combined with the ability to generate a variety of complex electrical waveforms (e.g., pulses), presents a huge selection of modulation parameter sets to the clinician or patient. For example, if the neuromodulation system to be programmed has sixteen electrodes, millions of modulation parameter sets may be available for programming into the neuromodulation system. To facilitate such selection, the clinician generally programs the modulation parameters sets through a computerized programming system to allow the optimum modulation parameters to be determined based on patient feedback or other means and to subsequently program the desired modulation parameter sets.

FIG. 9 illustrates, by way of example and not limitation, a system similar to the electrical therapy-delivery system of FIG. 8, which may be configured to provide DBS or SCS therapy(ies). The illustrated system 900 includes an external system 954 that may include at least one programming device. The illustrated external system 954 may include a clinician programmer 925 similar to CP 425 in FIG. 4, configured for use by a clinician to communicate with and program the neuromodulator, and a remote control device 924, similar to RC 424 in FIG. 4, configured for use by the patient to communicate with and program the neuromodulator. For example, the remote control device 924 may allow the patient to turn a therapy on and off, change or select programs, and/or may allow the patient to adjust patient-programmable parameter(s) of the plurality of modulation parameters. The monitor and/or therapy device may be implemented using an implantable medical device and/or an external device such as a wearable device. The external system 954 may include a network of computers, including computer(s) remotely located from the IMD (e.g., DBS or SCS) that are capable of communicating via one or more communication networks with the programmer 925 and/or the remote control device 924. The remotely located computer(s), such as cloud servers 955, and the IMD (e.g., DBS or SCS) may be configured to communicate with each other via another external device such as the programmer 925 or the remote control device 924. The remote control device 924 and/or the programmer 925 may allow a user (e.g., patient, caregiver and/or clinician or rep) to answer questions as part of a data collection process. The external system 954 may include personal devices such as a phone or tablet 956, wearables such as a watch 957, sensors or therapy-applying devices. The watch may include sensor(s), such as sensor(s) for detecting activity, motion and/or posture. Other wearable sensor(s) may be configured for use to detect activity, motion and/or posture of the patient. The cloud servers 955 may include data, such as medical record(s) for the patient and broader patient population(s) and may also include processing circuitry configured to develop and/or implement algorithm(s) for determining CVA alerts based at least in part on the sensed signals.

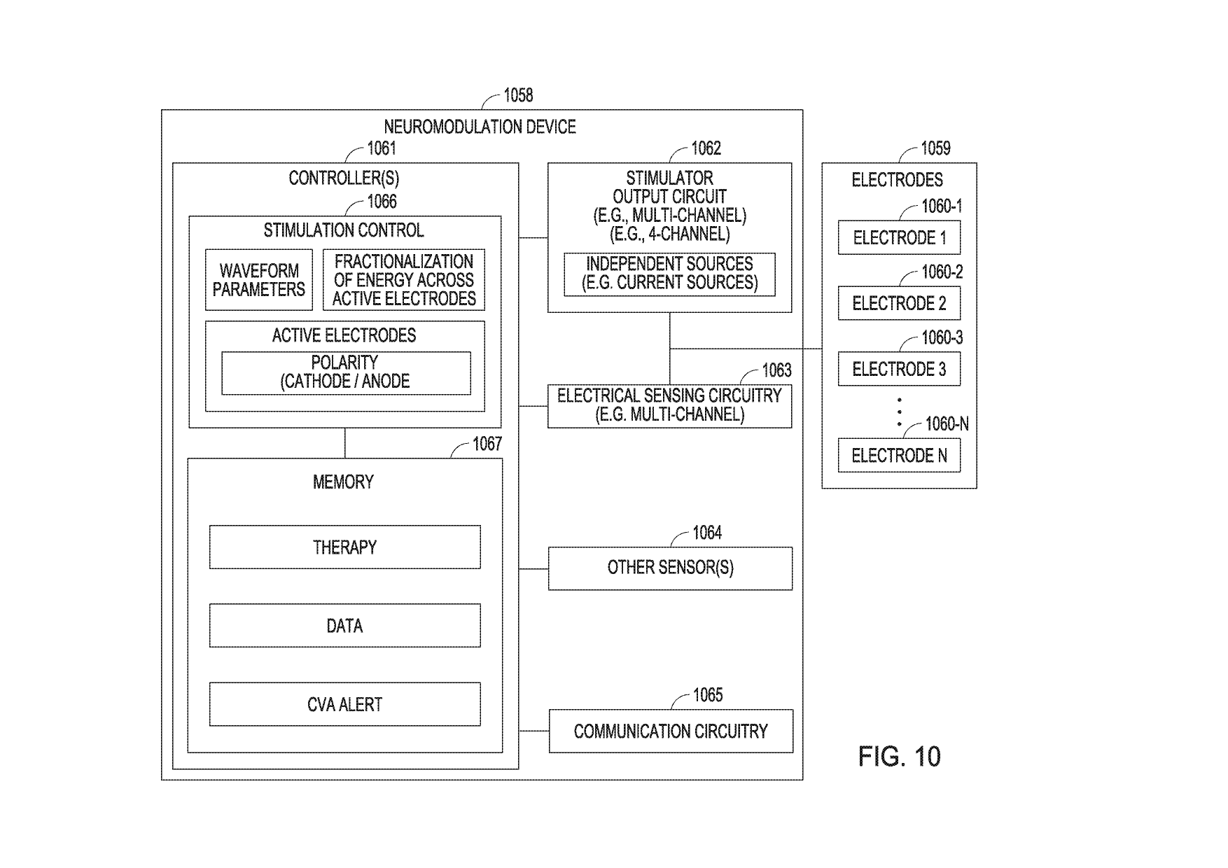

FIG. 10 illustrates, by way of example and not limitation, a neuromodulation device for delivering neuromodulation and for determining a likelihood of ongoing or imminent cerebrovascular accident (CVA) from sensed electrical signals and for communicating an alert. The neuromodulation device 1058 may correspond to the IPG 423 and/or the ETM 426 in FIG. 4, for example. The neuromodulation device 1058 may be connected to a lead system 1059 which includes one or more leads each configured to be electrically connected to the neuromodulation device 1058 and a plurality of electrodes 1060-1 to 1060-N distributed in an electrode arrangement using the one or more leads. The illustrated neuromodulation device 1058 includes one or more controllers 1061 operably connected to a stimulator output circuit 1062 to deliver neuromodulation to the electrodes. The stimulator output circuit 1062 may include a plurality of independent sources such as independent current sources for each electrode. The stimulator output circuit 1062 may be configured as a multi-channel (such as but not limited to four channels) system capable of simultaneously and independently generating and delivering separate stimulation waveforms to different electrode combinations. Some embodiments of the neuromodulation device 1058 may include electrical sensing circuitry 1063 configured to sense electrical activity (e.g., local field potentials, evoked compound actions potentials, evoked resonant neural activity (ERNA), electrospinogram, or other electrical signals) using at least some of the electrodes. Some embodiments of the neuromodulation device 1058 may include other sensor(s) 1064 that may be used to control the neuromodulation or provide context for the therapy or other events, or to detect events. Examples of other sensors include sensors configured to detect cardiac activity such as but not limited to heart rhythm, heart rate, pulse and blood flow, muscle activity, respiration such as but not limited to respiration rate and volume, activity, posture or electrical activity such as ecaps, LFPs or ERNA. The sensor(s) of the therapy device(s) may be used with the electrical neural activity sensors (e.g., LPF sensors or ECAP sensors) to detect CVA events, such as epileptic events, or strokes. Some embodiments of the neuromodulation device 1058 may include communication circuitry 1065 used to communicate with at least one external device. The controller(s) 1061 may be configured to provide stimulation control 1066 to control the neuromodulation generated by the stimulator output circuit 1062 and delivered to the electrodes, which may include the waveform parameters for the neuromodulation, the active electrodes and polarity of the active electrodes used to deliver the neuromodulation, and the fractionalization of energy across the active electrodes. The controller(s) 1061 may include memory 1067 configured to store data, configured to store therapy programs, and configured to implement process(es) used to provide CVA alerts based at least in part on the sensed electrical signals.

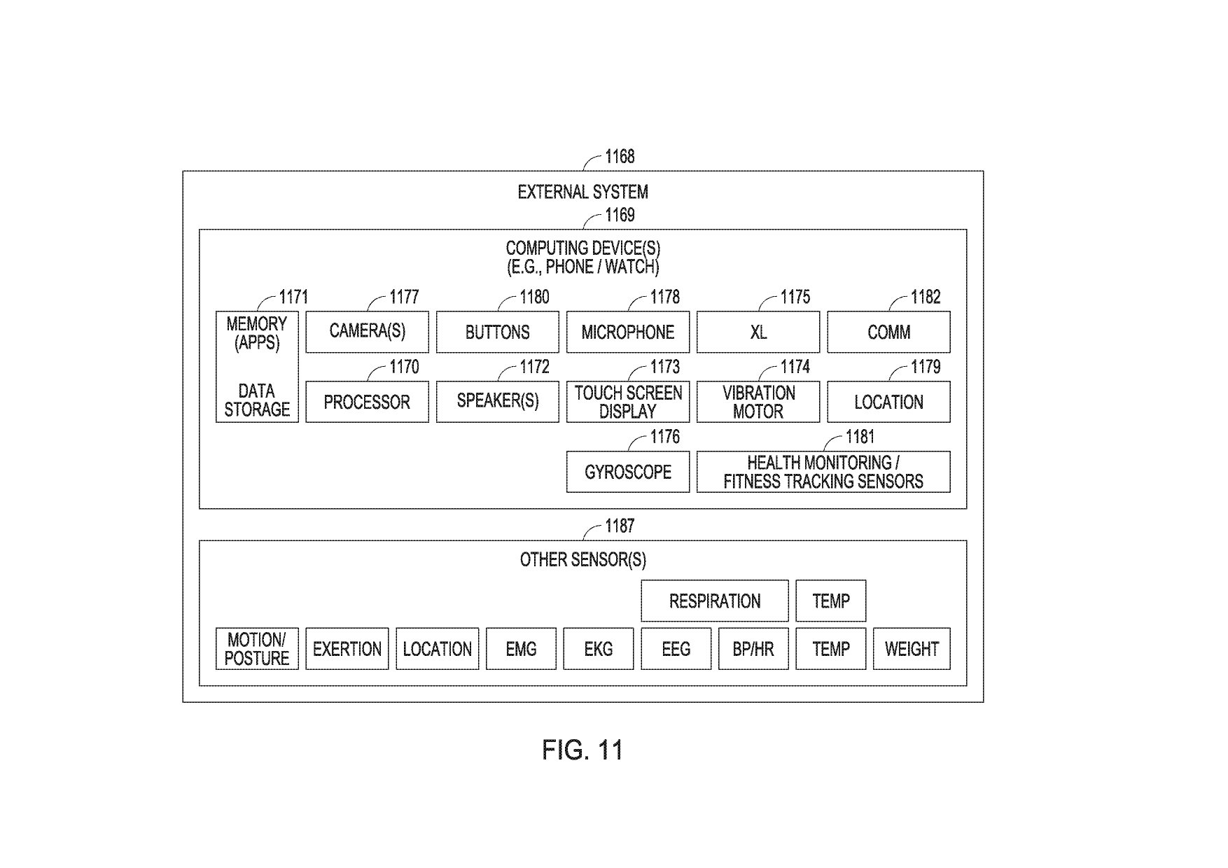

In addition to alternatively to using other sensors in the neurostimulator, some embodiments use sensor(s) in external systems to provide CVA alerts. FIG. 11 illustrates, by way of example and not limitation, an external system with sensing functions. The external system 1168 may include one or more computing devices 1169. The computing device(s) 1169 may include a phone, a tablet, and/or a wearable such as a watch, by way of example. The computing device(s) may include a number of features that may be used, alone or in combination with other features, by the system to detect events and/or respond to detected events. For example, the computing device(s) (e.g., phone, watch, and the like) may include a processor 1170 and a memory 1171 including apps to be implemented by the processor to perform various processes. The memory 1171 may provide data storage for the external system. Other features of the computing device(s) 1169 may include at least one of speaker(s) 1172 to produce acoustic signals, a touch screen display 1173 for providing a user interface used to receive user inputs and provide visual outputs, and a vibration motor 1174 that may be used to provide a haptic output. Some CVA alerts may involve outputting a haptic signal such as a pattern of vibrations. The computing device(s) 1169 may include feature(s) capable of being used alone or in various combinations to detect an event. For example, accelerometer(s) (XL) 111275, gyroscope 1176, camera(s) 1177, microphone(s) 1178 and/or location service(s) 1179 may be used, with or without a clock and information about the patient’s activities at different time(s) and/or location(s), to determine events. The location service(s) may use global positioning system (GPS), Wi-Fi and cellular towers (e.g., triangulation of Wi-Fi access points and/or cellular towers), Bluetooth beacons or Radio Frequency Identification (RFID). A determined location may be used to detect an event. For example, XLs 1175 can detect motion and/or posture. Microphones 1178 can be used to detect patient effort (breathing, grunts) and sound at the patient’s location that may be used to detect an event. Cameras 1177 may be used to detect motion, location, and patient effort, such as via a shaky image, facial feature(s) of the patient, visual detection of an epileptic event, bradykinesia event or other event. The touch screen display 1173 may be used to determine attempted use of the computing device (e.g., phone) via an unlocked screen or other interaction with the device via the touch screen display or other button(s) 1180 on the device, which may be used to detect an event. The computing device(s) 1169 may include health monitoring / fitness tracking sensor(s) 1181 that may make use of other sensor(s) in the device. Health monitoring / fitness tracking sensor(s) 1181 may be configured to detect at least an estimate of heart rate, ECG, blood pressure, oxygen, steps, sleep, exercise, stress, and the like. A more exhaustive list of sensor(s) of computing device(s) may include any one or various combinations of an accelerometer, a gyroscope, a magnetometer / compass, a barometric pressure sensor, a body temperature sensor, a heart rate monitor, an oximetry sensor, an ambient light sensor, a bioimpedance sensor, a proximity sensor, an orientation sensor, a pedometer, a calorie counter, an ECG sensor, a gesture sensor, a UV sensor, an electrodermal activity sensor, a skin conductance sensor, and a GPS sensor.

The computing device(s) 1169 in the external system 1168 also includes a number of features that may be used to detect events and/or respond to a detected event. For example, the computing device(s) 1169 may include communication technology 1182 (e.g., Wi-Fi, Bluetooth) for use to communicate with other computing device(s), other sensor(s), and/or other perceptible signal transducer(s). Other sensor(s) 1187 may include other motion, exertion and/or posture sensors, other exertion sensor(s), other sensor(s) for detecting location (e.g., beacon, such as within range of a Bluetooth device), other sensors of physiological parameters such as EEG, EMG, EKG, respiration, galvanic skin response (GSR), cardiovascular parameters such blood pressure, rhythm and/or heart rate, temperature, and weight. One or more of these sensors may be used, in conjunction with the sensed electrical signals, to detect CVA likelihood.

The computing device, such as may be used to program or control the operation of a medical device system, may include a processor, a memory, a display, and an input device. The computing device may be separate from and communicatively coupled to the therapy device and/or may be integrated with the therapy device. The functions associated with the computing device may be distributed among two or more devices, such that there may be two or more memory devices performing memory functions, two or more processors performing processing functions, two or more displays performing display functions, and/or two or more input devices performing input functions. In some examples, the computing device includes a watch, wristband, smartphone, or the like. Such computing devices can wirelessly communicate with the other components of the medical device system, such as the CP, RC, ETM, or IPG. The computing device may be used for gathering patient information, such as general activity level or present queries or tests to the patient to identify or score pain, depression, stimulation effects or side effects, cognitive ability, physical state or the like. In some examples, the computing device may prompt the patient to take a periodic test (for example, every day) for cognitive ability to monitor, for example, progression of a disease that can impair cognitive function or to help monitor for CVA events. In some examples, the computing device may detect, or otherwise receive as input, patient clinical responses to electrostimulation such as DBS, and determine or update stimulation parameters using a closed-loop algorithm based on the patient clinical responses. Examples of the patient clinical responses may include physiological signals (e.g., heart rate) or motor parameters (e.g., tremor, rigidity, bradykinesia). The computing device may communicate component(s) within the medical device system and may direct the changes to the stimulation parameters to one or more of those components. In some examples, the computing device can be a wearable device used by the patient only during programming sessions. Alternatively, the computing device can be worn all the time and continually or periodically adjust the stimulation parameters. In an example, a closed-loop algorithm for determining or updating stimulation parameters can be implemented in a mobile device, such as a smartphone, that is connected to the IPG or an evaluating device (e.g., a wristband or watch). These devices can also record and send information to the clinician.

The processor 1170 may include one or more processors that may be local to the user or non-local to the user or other components of the computing device. A stimulation setting (e.g., parameter set) includes an electrode configuration and values for one or more stimulation parameters. The electrode configuration may include information about electrodes (ring electrodes and/or segmented electrodes) selected to be active for delivering stimulation (ON) or inactive (OFF), polarity of the selected electrodes, electrode locations (e.g., longitudinal positions of ring electrodes along the length of a non-directional lead, or longitudinal positions and angular positions of segmented electrodes on a circumference at a longitudinal position of a directional lead), stimulation modes such as monopolar pacing or bipolar pacing, etc. The stimulation parameters may include, for example, current amplitude values, current fractionalization across electrodes, stimulation frequency, stimulation pulse width, etc. In various examples, portions of the functions of the processor may be implemented as a part of a microprocessor circuit. The microprocessor circuit can be a dedicated processor such as a digital signal processor, application specific integrated circuit (ASIC), microprocessor, or other type of processor for processing information. Alternatively, the microprocessor circuit can be a processor that can receive and execute a set of instructions of performing the functions, methods, or techniques described herein.

Computer models may be developed to analyze the sensed signal (e.g., LFPs in the brain or ecaps in or near the spinal cord) for the purpose of determining when there is a likelihood of an ongoing or an imminent CVA. These computer models may be created using machine learning and be developed and trained using the sensed electrical signal from the patient. Some embodiments may train the model using sensed electrical signals from larger patient populations.

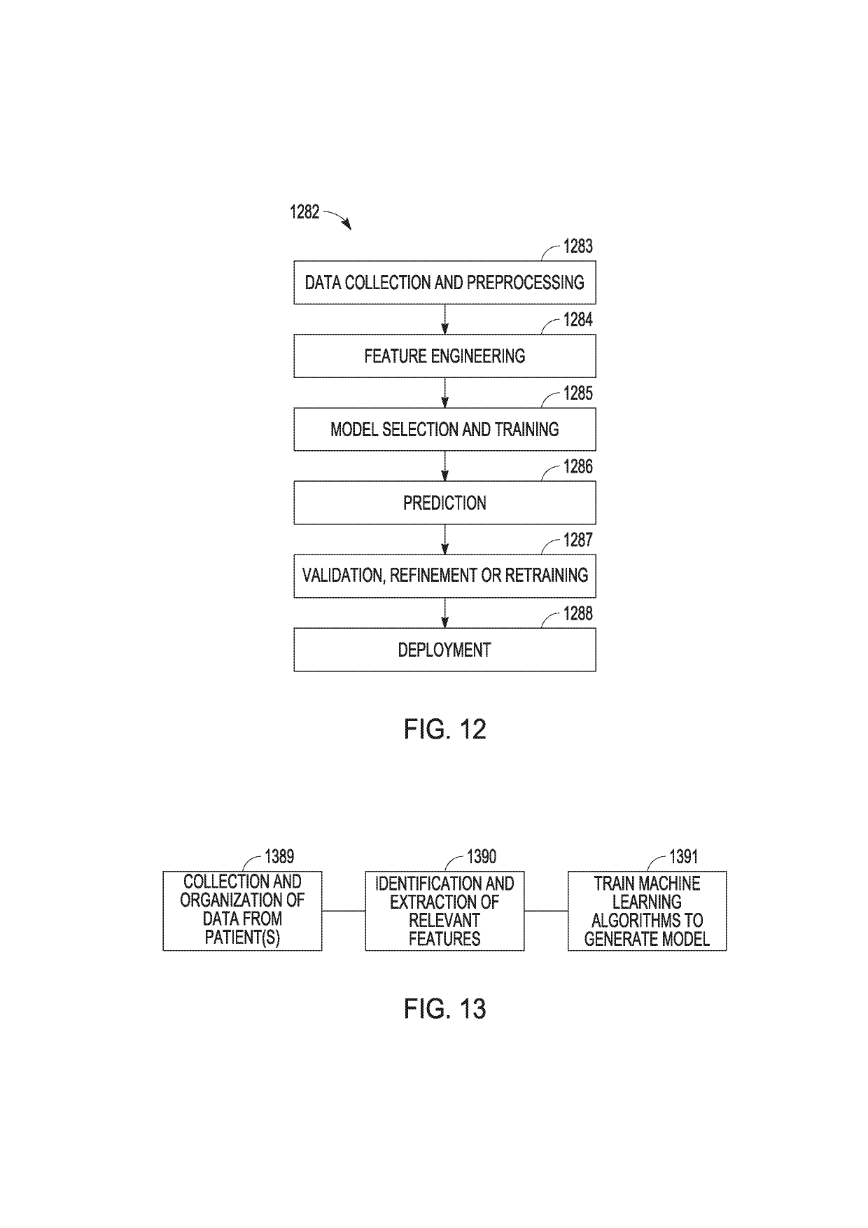

FIG. 12 is a flowchart depicting machine-learning pipeline, according to some examples. The machine-learning pipeline 1282 may be used to generate a trained model. Broadly, machine learning may involve using computer algorithms to automatically learn patterns and relationships in data, potentially without the need for explicit programming. Machine learning algorithms can be divided into three main categories: supervised learning, unsupervised learning, and reinforcement learning. Supervised learning involves training a model using labeled data to predict an output for new, unseen inputs. Examples of supervised learning algorithms include linear regression, decision trees, and neural networks. Unsupervised learning involves training a model on unlabeled data to find hidden patterns and relationships in the data. Examples of unsupervised learning algorithms include clustering, principal component analysis, and generative models like autoencoders. Reinforcement learning involves training a model to make decisions in a dynamic environment by receiving feedback in the form of rewards or penalties. Examples of specific machine learning algorithms that may be deployed, according to some examples, include various types of regression algorithms, which are a type of supervised learning algorithm. The performance of machine learning models is typically evaluated on a separate test set of data that was not used during training to ensure that the model can generalize to new, unseen data. Regression algorithms aim at quantifying some items (for example, by providing a value that is a real number). Generating a trained machine-learning program may include multiple phases that form part of the machine-learning pipeline 1282. A data collection and preprocessing 1283 phase may include acquiring and cleaning data to ensure that it is suitable for use in the machine learning model. This phase may also include removing duplicates, handling missing values, and converting data into a suitable format. A feature engineering 1284 phase may include selecting and transforming the training data to create features that are useful for predicting the target variable. Feature engineering may include receiving features (e.g., as structured or labeled data in supervised learning) and/or identifying features (e.g., unstructured, or unlabeled data for unsupervised learning) in training data. A model selection and training 1285 phase may include selecting an appropriate machine learning algorithm and training it on the preprocessed data. This phase may further involve splitting the data into training and testing sets, using cross-validation to evaluate the model, and tuning hyperparameters to improve performance. A model evaluation 2004 may include evaluating the performance of a trained model on a separate testing dataset. This phase can help determine if the model is overfitting or underfitting and determine whether the model is suitable for deployment. A prediction 1286 involves using a trained model to generate predictions on new, unseen data. A validation, refinement or retraining 1287 phase may include updating a model based on feedback generated from the prediction phase, such as new data or user feedback. A deployment 1288 phase may include integrating the trained model into an application or a more extensive system.

FIG. 13 illustrates, by way of example and not limitation, a process for creating machine-learning models that may be used to determine a likelihood of an ongoing or an imminent CVA and to compare the determined likelihood to a predefined threshold for a CVA alert. At 1389, previous electrical sensing sessions from multiple patients may be collected and organized, similar to data collection and preprocessing 1283 in FIG. 12. At 1390, relative features from the data may be identified and extracted, similar to feature engineering 1284 in FIG. 12. At 1391, these features from the collected data may be used to train the machine learning algorithm(s) to generate regression models, which may be similar to one or more of model selection and training 1391, model evaluation 1392, and validation, refinement or retraining 1393.

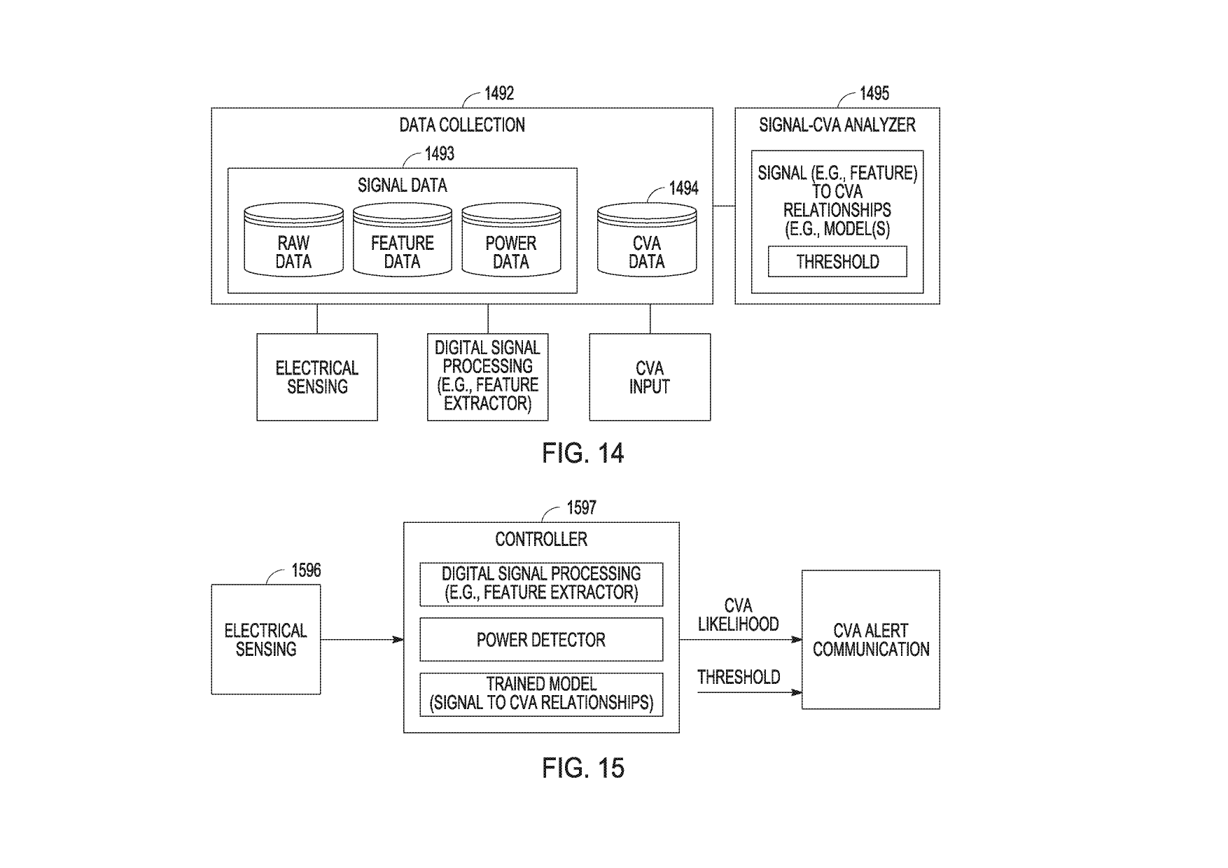

FIG. 14 illustrates, by way of example and not limitation, training of a machine-learning program to detect the CVA likelihood, according to some example embodiments. Machine-learning programs (MLPs), also referred to as machine-learning algorithms or tools, are utilized to perform operations associated with machine learning tasks such as identifying relationship(s) in the collected data. Machine learning (or other artificial intelligence) may be implemented to identify event(s) that appear to have useful for determining a likelihood of an ongoing or an imminent CVA and provide a CVA alert. For example, machine learning may identify feature(s) within the sensed signal that are indicative of the event(s), either by themselves or with other inputs, that are useful for determining a likelihood of an ongoing or an imminent CVA and provide a CVA alert. The system may include a data collection system 1492 configured to detect signal data 1493 (e.g., raw data for the sensed signal, feature data for the sensed signals and/or power data for the sensed signals) and CVA data 1494. The data may be collected directly from the raw sensed electrical signals or from processed electrical signals. The signal may be processed to pass a desired frequency range (or ranges) for further processing. For example, a signal power for specific frequencies may be determined and monitored, and a change in the power in the signal at certain frequencies that is greater than a threshold may indicate a potential CVA event. Digital signal processing, which may also be referenced as a feature extractor, may be used to extract or determine feature(s) found in the sensed electrical signals. CVA input may be used to identify CVA or potential CVA events such as a potential seizure or potential stroke. The CVA inputs may be user inputs into the training of the machine learning program to indicate when a patient experienced a seizure or stroke. The signal-CVA analyzer 1495 may be configured to use machine learning (or other artificial intelligence) to analyze the collected data and CVA data to identify signal-to-CVA relationship(s) (e.g., develop models) that may be used to determine likelihood of an ongoing or an imminent CVA and provide a CVA alert. A plurality of signals may be detected to compile signal data. The signal CVA analyzer 1495 may operate to develop a programming model by training, re-training, or updating a model (e.g., an artificial intelligence model, such as a neural network) based on the analyzed data. One or multiple instances of a model may be trained to generate programs and program parameters, for any one or more of two or more therapies. The models may be patient-specific or may be developed for a larger population.

More particularly, machine learning is a field of study that gives computers the ability to learn without being explicitly programmed. Machine learning explores the study and construction of algorithms that may learn from existing data (e.g., “training data”) and make predictions about new data. Such machine-learning tools may build a model from example training data in order to make data-driven predictions or decisions expressed as outputs or assessments. The machine-learning algorithms use the training data to find correlations among identified features that affect the outcome. The machine-learning algorithms use features for analyzing the data to generate assessments. Digital signal processing may be used to extract feature(s) within the sensed electrical signal. A feature is an individual measurable property of the observed phenomenon. Detected feature(s) may be defined within a frequency domain and/or a time domain. In the context of a biological signal, some examples of features may include, but are not limited to, peak(s) such as a minimum peak, a maximum peak as well as local minimum and maximum peaks, a range between peaks, a difference in values for features, a feature change with respect to a baseline, an area under a curve, a curve length, an oscillation frequency, and a rate of decay for peak amplitude. Inflection points in the signal may also be an observable feature of the signal, as an inflection point is a point where the signal changes concavity (e.g., from concave up to concave down, or vice versa), and may be identified by determining where the second derivative of the signal is zero. The system may analyze a feature change with respect to a baseline, a number of samples or time between consecutive local minimums and maximums, an area under the curve, a rate of decay for a peak amplitude, a firmware calculated Fast Fourier Transform (FFT), and a correlation strength between magnitude and phase across frequency bands of signal. A custom ASIC / hardware / firmware is capable of extracting some features. Software is capable of extracting a large number of features. Detected feature(s) may be partially defined by time (e.g., length of curve over a time duration, area under a curve over a time duration, maximum or minimum peak within a time duration, etc.). The machine-learning algorithms may use the training data to find correlations among the identified features that affect the outcome or assessment. The machine-learning tool may be trained using the training data and identified feature(s). The machine-learning tool may appraise the value of the features as they correlate to the training data. The result of the training is the trained machine-learning program.

Various machine learning techniques may be used to train models to make predictions based on data fed into the models. During a learning phase, the models may be developed against a training dataset of inputs to optimize the models to correctly predict the output for a given input. A training data set may be defined for desired functionality of the closed-loop algorithm and closed loop parameters may be defined for desired functionality of the closed-loop algorithm. Generally, the learning phase may be supervised, semi-supervised, or unsupervised; indicating a decreasing level to which the “correct” outputs are provided in correspondence to the training inputs. In a supervised learning phase, all of the outputs may be provided to the model and the model may be directed to develop a general rule or algorithm that maps the input to the output. In contrast, in an unsupervised learning phase, the desired output is not provided for the inputs so that the model may develop its own rules to discover relationships within the training dataset. In a semi-supervised learning phase, an incompletely labeled training set may be provided, with some of the outputs known and some unknown for the training dataset. Models may be run against a training dataset for several epochs (e.g., iterations), in which the training dataset is repeatedly fed into the model to refine its results. For example, in a supervised learning phase, a model may be developed to predict the output for a given set of inputs and may be evaluated over several epochs to more reliably provide the output that is specified as corresponding to the given input for the greatest number of inputs for the training dataset. In another example, for an unsupervised learning phase, a model may be developed to cluster the dataset into groups and is evaluated over several epochs as to how consistently it places a given input into a given group and how reliably it produces the desired clusters across each epoch.

Once an epoch is run, the models may be evaluated, and the values of their variables may be adjusted to attempt to better refine the model in an iterative fashion. In various aspects, the evaluations may be biased against false negatives, biased against false positives, or evenly biased with respect to the overall accuracy of the model. The values may be adjusted in several ways depending on the machine learning technique used. For example, in a genetic or evolutionary algorithm, the values for the models that are most successful in predicting the desired outputs are used to develop values for models to use during the subsequent epoch, which may include random variation/mutation to provide additional data points. One of ordinary skill in the art will be familiar with several machine learning algorithms, including linear regression, random forests, decision tree learning, neural networks, deep neural networks, and the like.

FIG. 15 illustrates, by way of example and not limitation, use of a system configured to detect the CVA likelihood, according to some example embodiments. At 1696, the system senses the electrical signals. For example, sensing the electrical signals may include sensing LFPs using a DBS system or sensing ecaps using a an SCS system. The sensing may be performed using the electrical sensing circuitry 103 and a sensing configuration 160 of the electrodes 104 within the medical device system illustrated in FIG. 1. At 1597, a controller, which may be part of a processing system, may analyze the sensed electrical signal to determine a likelihood of ongoing or imminent CVA. At 1598, the likelihood of ongoing or imminent CVA may be compared to a threshold (or thresholds or another baseline), which may be used to determine when to provide communicate a CVA alert.

The controller may perform one or more processes on the sensed electrical signal. The processes may include digital signal processing, which may be used to extract one or more features of the sensed electrical signal. A value or values of the feature(s) may be compared to a threshold to determine a likelihood of a CVA event. The processes may include a power detector which may detect a power for the signal (or select frequency range(s) of the signal). Brain signals, for example, may be decomposed into delta (1–4 Hz), theta (4–8 Hz), alpha (8–13 Hz), beta (13–30 Hz), gamma (30–70 Hz), and high gamma (70–120 Hz). Fast Fourier Transform (FFT) may be used to convert the time-domain signals into the frequency domain, and power may correspond to a magnitude of the signal within a specific frequency band. Power level(s) may be compared to threshold(s) and used to determine a likelihood of a CVA event and to determine when to provide the CVA alert. The threshold(s) may be identified for a patient, or group of patients, during baseline times without a CVA event (e.g., seizure or stroke). Significant changes in the power from baseline times may indicate a likelihood of a CVA event. Certain patterns of power changes in the different frequency ranges also may be used to identify a likelihood of a CVA event.

The controller may implement a trained computerized model that implements a signal to CVA relationship. A likelihood of a CVA event may be determined from the trained model, which may be compared to a threshold to determine when to provide the CVA alert.

The trained computerized model may be any suitable trained computerized model, such as a neural network. A neural network is a type of computerized model that is constructed from interconnected cells divided into layers. Each cell is implemented as a function executed by the sensor electronics or other processor or computing system. The sensor electronics or other system implements a cell by processing cell input data and generating corresponding cell output data according to the cell’s function. In some examples, the cells of the neural network are arranged in layers. An input layer of cells receives an input to the neural network. The inputs to cells making up the intermediate layers may include outputs of cells in the input layer and/or outputs of other cells in the intermediate layers. Cells in an output layer receive inputs generated, for example, by cells in the intermediate layers and generate outputs. The outputs of the cells of the output layer may represent the sensor decline metric or other output of the neural network.

A neural network may be trained utilizing labeled training data to determine a likelihood of an ongoing or an imminent CVA and provide a CVA alert. Labeled training data comprises instances of input data and a corresponding output. In the context of determining a likelihood of an ongoing or an imminent CVA, for example, labeled training data may comprise sensed electrical signals and CVA event labels. Training may be conducted in a series of training epochs. In each training epoch, some or all of the training data may be provided as input to the neural network. The output of the neural network may be compared to the corresponding label or labels to determine a loss of the neural network. The loss describes a difference between the output of the neural network and the label or labels indicating the correct output for the corresponding input. The loss is used to modify the neural network, for example, by modifying weights or other portions of the functions implemented by the individual cells. In some examples, a predetermined number of training epochs are performed. Also, in some examples, training epochs are performed until the loss of the neural network meets a threshold. Various different techniques may be used to determine the loss and to determine updates to neural network cells during a training epoch. Example techniques include gradient descent and back propagation.

Some embodiments may use artificial intelligence architecture to classify and determine a likelihood of an ongoing or an imminent CVA, such as a Convolutional Neural Network (CNN), a Recurrent Neural Network (RNN), a Long-Short Term Memory (LSTM) network, or a transformer. A CNN is a unidirectional or feed forward neural network in which the inputs to the cells are based on the outputs of cells from prior layers. An RNN is a bidirectional neural network. For example, some cells of the RNN may receive input that is based on the outputs of previous cells and may, additionally or alternatively, receive input that depends on a previous output of the same cell. An example RNN is an LSTM or LSTM layer. Transformers do not rely on recurrence but instead operate on self-attention.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

Method examples described herein may be machine or computer-implemented at least in part. Some examples may include a computer-readable medium or machine-readable medium encrypted with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media (referred to herein as computer readable medium), such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like. The term "machine" may include at least one processor/controller, including one processor/controller to implement all of the instructions, at least two processors/controllers where one processor/controller operates on some of the instructions and other processor(s)/controller(s) operate on other instructions, or at least two processors/controllers where each processor/controller is capable of operating on the same instructions. Thus, for example, distributed systems or systems with shared resources are contemplated.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A method implemented using an implantable medical device having electrical stimulation circuitry, electrical sensing circuitry and a set of electrodes from which to provide a stimulation electrode configuration and a sensing electrode configuration, wherein the method comprises:
delivering a therapy by delivering electrical stimulation using the electrical stimulation circuitry of the implantable medical device, wherein the electrical stimulation is delivered using the stimulation electrode configuration;
sensing electrical signals using the electrical sensing circuitry of the implantable medical device, wherein the electrical signals are sensed using the sensing electrode configuration;
using a processing system to analyze the sensed electrical signals to determine a likelihood of an ongoing or an imminent cerebrovascular accident (CVA) and to compare the determined likelihood to a predefined threshold to provide a comparison; and
communicating, using a communication system, an alert based on the comparison of the determined likelihood to the predefined threshold.

2. The method of claim 1, wherein the CVA includes a stroke and the therapy is not a therapy to either prevent or treat stroke.

3. The method of claim 1, wherein the CVA includes a seizure, and the therapy is not a therapy to either prevent or treat the seizure.

4. The method of claim 1, wherein the therapy includes a DBS therapy and the electrical signals sensed using the electrical sensing circuitry includes local field potentials in a brain.

5. The method of claim 4, wherein the DBS therapy includes a DBS therapy for a movement disorder.

6. The method of claim 1, wherein the therapy includes a SCS therapy and the electrical signals sensed using the electrical sensing circuitry includes evoked compound action potentials.

7. The method of claim 1, wherein the system includes an external device, the processing system includes processing circuitry in the implantable medical device and in the external device, and the communication system includes wireless communication circuitry in the implantable medical device and wireless communication circuitry in the external device, wherein the method includes using the processing system and the communication system to communicate data corresponding to the likelihood or data used to determine the likelihood.

8. The method of claim 7, further comprising using the processing system and the communication system to alert a user of the external device based on the comparison of the determined likelihood to the predefined threshold.

9. The method of claim 7, further comprising using the processing system and the communication system to notify first responders, family members, or healthcare providers based on the comparison of the determined likelihood to the predefined threshold.

10. The method of claim 1, further comprising using the processing system to analyze an output of at least one of an accelerometer, a heart rate sensor, a blood pressure sensor or an oxygen sensor with the sensed electrical signals to determine a likelihood of ongoing or imminent CVA.

11. The method of claim 1, wherein the sensed signals are analyzed by applying digital signal processing or using a trained neural network to the sensed electrical signals to determine the likelihood of ongoing or imminent CVA, or are analyzed by analyzing power changes in the sensed signals to determine the likelihood of ongoing or imminent CVA.

12. The method of claim 1, further comprising training a model to determine the likelihood of ongoing or imminent cerebrovascular accident (CVA), wherein the model is trained by:
receiving signal data corresponding to sensed electrical signals from one or more patients;
receiving ongoing or imminent CVA data for the one or more patients;
determining patterns indicative of CVA including determining one or more features within the sensed electrical signals that are capable of being used to determine the likelihood of ongoing or imminent CVA;
determining one or more feature-CVA relationships associating the one or more features within the sensed electrical signals to the likelihood of ongoing or imminent CVA;
updating the one or more feature-CVA relationships based on additional signal data and additional CVA data; and
adjusting the predefined threshold.

13. A method for modifying a medical device system that includes a cloud server, an external device configured to communicate with the cloud server, and an implantable medical device configured to communicate with the external device, the implantable medical device having electrical stimulation circuitry, electrical sensing circuitry and a set of electrodes from which to provide a stimulation electrode configuration and a sensing electrode configuration, wherein the implantable medical device is configured to deliver spinal cord stimulation (SCS) therapy to treat pain or is configured to deliver deep brain stimulation (DBS) to treat a movement disorder, the method comprising:
programming the medical device system to sense electrical signals using the electrical sensing circuitry and the sensing electrode configuration;
programming the medical device system to analyze the sensed electrical signals to determine a likelihood of an ongoing or an imminent cerebrovascular accident (CVA);
programming the medical device system to compare the determined likelihood to a predefined threshold to provide a comparison; and
programming the medical device system to communicate, based on the comparison, an alert through at least one of the external device or the cloud server to notify first responders, family members, or healthcare providers.

14. The method of claim 13, wherein the method includes updating firmware in the implantable medical device.

15. The method of claim 13, wherein the method includes updating or installing an app in the external device.

16. The method of claim 13, wherein the medical device system is programmed to perform digital signal processing to the sensed electrical signals to determine the likelihood of ongoing or imminent CVA.

17. The method of claim 13, wherein the medical device system is programmed to use a trained neural network to determine the likelihood of ongoing or imminent CVA.

18. The method of claim 13, wherein the medical device system is programmed to analyze power changes in the sensed signals to determine the likelihood of ongoing or imminent CVA.

19. The method of claim 13, wherein the medical device system is programmed to analyze an output of at least one of an accelerometer, a heart rate sensor, a blood pressure sensor or an oxygen sensor, along with the sensed electrical signals, to determine a likelihood of ongoing or imminent CVA.

20. A system, comprising:
an implantable medical device configured for implantation in a patient, the implantable medical device including:
a set of electrodes from which to provide a stimulation electrode configuration and sensing electrode configuration;
electrical stimulation circuitry configured to deliver a therapy by delivering electrical stimulation using the stimulation electrode configuration; and
electrical sensing circuitry configured to use the sensing electrode configuration to sense electrical signals;
a processing system configured to analyze the sensed electrical signals to determine a likelihood of ongoing or imminent cerebrovascular accident (CVA) and configured to provide a comparison of the determined likelihood to a predefined threshold; and
a communication system, wherein the processing system is configured to use the communication system to communicate an alert based on the comparison of the determined likelihood to the predefined threshold.
